(19) United States
(12) Patent Application Publication
(10) Pub. No.: US 2010/0135855 A1
(43) Pub. Date: Jun. 3, 2010

(54) METHOD FOR DEPOSITING SUBSTANCES ON A SUPPORT

(75) Inventors: ANKE PIERIK, EINDHOVEN (NL); JOHAN FREDERIK DIJKSMAN, EINDHOVEN (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/625,803

(22) Filed: Nov. 25, 2009

(30) Foreign Application Priority Data
Nov. 26, 2008 (EP) 08169968.8

(51) Int. Cl. G01N 33/48; B05D 1/36

(52) U.S. Cl. 422/68.1; 427/261

(57) ABSTRACT

The present invention relates to a method for depositing a substance on a support, comprising the provision of a substance solution, the provision of a transfer solution capable of activating the support, the deposition of the transfer solution on a predefined position of the support and the deposition of the substance solution on the same predefined position where the transfer solution was placed, whereby an immobilization of the deposited substance at the location of overlap between the deposited transfer solution and the deposited substance solution on said support is achieved.

The present invention further relates to the use of a method for depositing a substance on a support for the manufacturing of a chip, a method for manufacturing a chip, wherein a substance is deposited on a chip substrate according to the method for depositing a substance on a support and a chip manufactured according to said method.

### FIELD OF THE INVENTION

The present invention relates to a method for depositing a substance on a support, comprising the provision of a substance solution, the provision of a transfer solution capable of activating the support, the deposition of the transfer solution on a predefined position of the support and the deposition of the substance solution on the same predefined position where the transfer solution was placed, whereby an immobilization of the deposited substance at the location of overlap between the deposited transfer solution and the deposited substance solution on said support is achieved.

The present invention further relates to the use of a method for depositing a substance on a support for the manufacturing of a chip, a method for manufacturing a chip, wherein a substance is deposited on a chip substrate according to the method for depositing a substance on a support and a chip manufactured according to said method.

### BACKGROUND OF THE INVENTION

Chips or microarrays comprising a multitude of substances, in particular biochips and DNA microarrays, have become an important tool in modern chemistry, molecular biology and medicine. Typically the chips consist of an arrayed series of a large number of microscopic spots of substances like nucleic acid molecules, each containing small amounts of a specific nucleic acid sequence. This can be, for example, a short section of a gene or other DNA element that are used as capture probes to hybridize a cDNA or cRNA sample (a target or target probe) under conditions, which allow a binding between the capture probe and the corresponding target. Capture probe-target hybridization is typically detected and quantified by fluorescence-based detection of fluorophore-labeled targets to determine relative abundance of nucleic acid sequences in the target.

Microarray technology evolved from Southern blotting, where fragmented DNA is attached to a substrate and then probed with a known gene or fragment. The use of a collection of distinct DNAs in arrays for expression profiling was first described in 1987, and the arrayed DNAs were used to identify genes whose expression is modulated by interferon. These early gene arrays were made by spotting cDNAs onto filter paper with a pin-spotting device. The use of miniaturized microarrays, in particular for gene expression profiling was first reported in the 1990s. A complete eukaryotic genome on a microarray was published in 1997.

A variety of technologies may be used in order to fabricate such microarrays. The techniques include printing with fine-pointed pins, photolithography using pre-made masks, photolithography using dynamic micromirror devices, ink jet printing (Lausted C. et al., 2004, Genome Biology 5: R58), or electrochemistry.

The photolithographic technique is directed to the production of oligonucleotide arrays by synthesizing the sequences directly onto the array surface. The technique involves photolithographic synthesis on a silica substrate where light and light-sensitive masking agents are utilized to generate a sequence one nucleotide at a time across the entire array (Pease et al., 1994, PNAS 91: 5022-5026). Each applicable probe is selectively unmasked prior to bathing the array in a solution of a single nucleotide, then a masking reaction takes place and the next set of probes are unmasked in preparation for a different nucleotide exposure. After several repetitions, the sequences of every probe become fully constructed. Accordingly constructed oligonucleotides may be longer (e.g. 60-mers) or shorter (e.g. 25-mers) depending on the desired purpose.

In spotted microarrays, the substances are deposited as intact substances, for instance the nucleic acids are synthesized prior to deposition on the array surface and are then spotted onto the substrate. A common approach utilizes an array of fine pins or needles controlled by a robotic arm that is dipped into wells containing, e.g., DNA probes and then depositing each probe at designated locations on the array surface, or an ink jet printing device, which deposits the probe material via the ejection of droplets. The resulting array of probes represents, for example, the nucleic acid profiles of a prepared capture probe and can interact with complementary cDNA or cRNA target probes, e.g. derived from experimental or clinical samples. In addition, these arrays may be easily customized for specific experiments, since the substances and printing locations on the arrays can be chosen specifically.

The control, adjustment and fine-tuning of spotting and deposition processes for the production of microarrays has been described, for example, in GB 2355716.

However, during the deposition and immobilization process a substance to be deposited may become subject to deflecting local forces when landing on a support material. For example, a drop of substance solution being ejected, for instance, from an ink jet printing device may splash when impacting on a support material. Such a splattering interaction with the material normally leads to the generation of satellite drops of the deposited substance, which may contribute to a decreased accuracy of the deposition process. Also satellite drops which are produced directly after the main droplet during the ink jet printing process lead to random small spots on the surface.

There is, thus, a need for a depositing method which allows an efficient and accurate deposition and immobilization of a substance on a support that overcomes the disadvantageous generation of satellite drops.

### OBJECTS AND SUMMARY OF THE INVENTION

The present invention addresses this need and provides means and methods which allow the accurate deposition of substances on a support.

The above objective is accomplished by a method for depositing and immobilizing a substance on a support, comprising the use of a transfer solution capable of activating the support and the corresponding deposition of the transfer solution on a predefined position of the support where the transfer solution was placed, whereby an immobilization of the deposited substance at the location of overlap between the deposited transfer solution and the deposited substance solution on said support is achieved, with the proviso that said transfer solution and said substance solution are not placed together or as a mixed solution on the support.

It is an advantage of the method according to the present invention that the positional accuracy of immobilized substance spots on a substrate is greatly improved. In particular, in case that satellite drops of a substance solution land on a support next to the main spot, these satellite drops will not be immobilized onto the substrate due to the lack of presence of transfer solution outside of the main spot. An additional advantage of the method of the invention is the concomitant reduction in size of the deposited substance dots, which spread only within the limited boundaries defined by the presence of the transfer solution. Furthermore, the method of the invention allows to reduce the amount of transfer solution needed to activate a support material in comparison to traditional activation processes, which activate the entire material. Moreover, according to the method of the invention, only a localized, spatially confined and, thus, economical activation of the support material is necessary in order to effectuate an efficient immobilization and the use of spatially localized transfer solutions for the deposition of substances allows to accurately define and adjust the period of time between the activation and deposition/immobilization of substances. This possibility contributes to a reduction of variation between sequentially spotted substances on a support.

In a preferred embodiment of the present invention, the interim between the deposition step of the transfer solution and the substance solution and vice versa is a predefined, fixed period of time.

In another preferred embodiment of the present invention the deposition of the substance solution of is carried out before the deposition of the transfer solution.

In a further preferred embodiment of the present invention, said support as mentioned above comprises amine-reactive groups.

In another preferred embodiment of the present invention, said support as mentioned above comprises carboxylic groups.

In a further preferred embodiment of the present invention, said support as mentioned above comprises a porous substrate. In a more preferred embodiment said above mentioned porous substrate is nylon

In yet another preferred embodiment of the present invention, said support as mentioned above comprises a non-porous substrate. In a more preferred embodiment of the present invention said non-porous substrate is composed of glass, poly-L-lysine coated material, nitrocellulose, polystyrene, cyclic olefin copolymer (COC), cyclic olefin polymer (COP), polypropylene, polyethylene or polycarbonate.

In a further preferred embodiment of the present invention, said activation of the support as mentioned above is a chemical activation

In yet another preferred embodiment of the present invention, said transfer solution as mentioned above comprises chemical moieties capable of reacting with amine groups or carboxylic groups.

In a particularly preferred embodiment of the present invention, said transfer solution as mentioned above comprises EDC (1-ethyl-3-(3-dimethylaminopropyl) carbodiimide hydrochloride) or NHS (N-hydrosuccinimide) or a mixture of EDC and NHS.

In another, preferred embodiment of the present invention the substance solution as mentioned above comprises a nucleic acid, a protein or a sugar, or a modified derivative thereof, or any combination thereof. In a particularly preferred embodiment, said substance solution comprises a nucleic acid, a protein or a sugar comprising an amine end group, or a modified derivative of a nucleic acid, a protein or a sugar comprising an amine group, e.g. an amine end group.

In a further preferred embodiment of the present invention, said method for depositing a substance on a support as mentioned above comprises a further step wherein the support is washed, whereby substance solution, which is not fixated at the location of overlap between the deposited transfer solution and the deposited substance solution on said support, is removed.

In a further aspect the present invention relates to the use of a method for depositing a substance on a support as mentioned above for the manufacturing of a chip.

In a further aspect the present invention relates to a method for manufacturing a chip, wherein a substance is deposited on a chip substrate according to the method for depositing a substance on a support as mentioned above.

In a further aspect the present invention relates a chip manufactured according to the method for depositing a substance on a support as mentioned above.

In a further preferred embodiment of the present invention the chip manufactured according to the method for depositing a substance on a support as mentioned above, is a packaged chip comprising a reaction chamber with inlets for flowing fluid, and alignment structures for placing the chip at a desired location with respect to a scanner.

These and other characteristics, features and objectives of the present invention will become apparent from the following detailed description, taken in conjunction with the accompanying figures and examples, which demonstrate by way of illustration the principles of the invention.

The description is given for the sake of example only, without limiting the scope of the invention.

### DESCRIPTION OF THE FIGURES

FIG. 1 shows a sample of spot pattern, printed on a porous substrate.

FIG. 2 depicts a schematic of a cross-section of a membrane comprising dots of deposited substances.

FIG. 3 depicts the formation of an amide using a carbodiimide. The following reaction steps are indicated in the figure: acid 1 reacts with the carbodiimide to produce key intermediate O-acylisourea 2, which can be viewed as a carboxylic ester with an activated leaving group. O-acylisourea will subsequently react with amines to give rise to amide 3 and urea 4. A side reaction of O-acylisourea 2 may give rise to different products. For example, O-acylisourea 2 may react with an additional carboxylic acid 1 to produce an acid anhydride 5, which can produce amide 3. A further, minor pathway involves the rearrangement of O-acylisourea 2 to stable N-acylurea 6.

FIG. 4 depicts the formation of an amide based on the use of EDC and Sulfo-NHS. EDC reacts with a carboxyl group on molecule 1, forming an amine-reactive O-acylisourea intermediate. This intermediate may react with an amine on molecule 2, yielding a conjugate of the two molecules joined by a stable amide bond. Since the intermediate is also susceptible to hydrolysis, is unstable and short-lived in aqueous solution. The addition of Sulfo-NHS stabilizes the amine-reactive intermediate by converting it to an amine-reactive Sulfo-NHS ester, thereby increasing the efficiency of EDC-mediated coupling reactions. The amine-reactive Sulfo-NHS ester intermediate is sufficiently stable to permit a two-step crosslinking procedure, which allows the carboxyl groups on one molecule to remain unaltered.

FIG. 5 shows a print layout for a spotting experiment, wherein reference numbers 1 denote spots where the transfer fluid has been deposited and the membrane is locally activated. Reference number 3 designates a fluorescently labeled oligonucleotide, which is used for positioning the grid over the spots.

FIG. 6 depicts a print layout for a spotting experiment, wherein reference numbers 1 denote spots where a fluorescently labeled capture probe was printed. Reference number 3 designates a fluorescently labeled oligonucleotide, which is used for positioning the grid over the spots. The fluorescently labeled capture probe was printed not only on the activated spots, but also on the columns in between references numbers 1 of FIG. 5.

FIG. 7 depicts an image of a membrane after printing of fluorescently labeled capture probes. The intensity of the spots is roughly equal due to the fact that the same number of fluorophores has been deposited on each spot. The spots, where the transfer fluid has been printed are smaller.

FIG. 8 depicts an image which was taken after the membrane shown in FIG. 7 was subjected to a washing step in order to remove all material that was not immobilized on the support. The spots, where the transfer fluid has been printed, are clearly visible, whereas the spots where no transfer fluid has been printed are hardly or not visible.

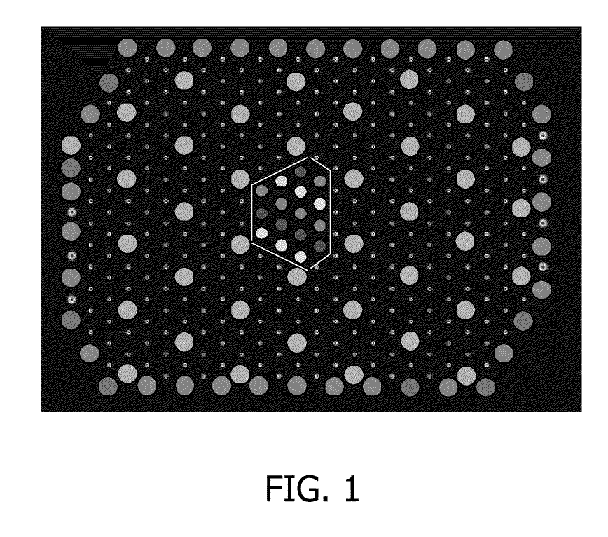

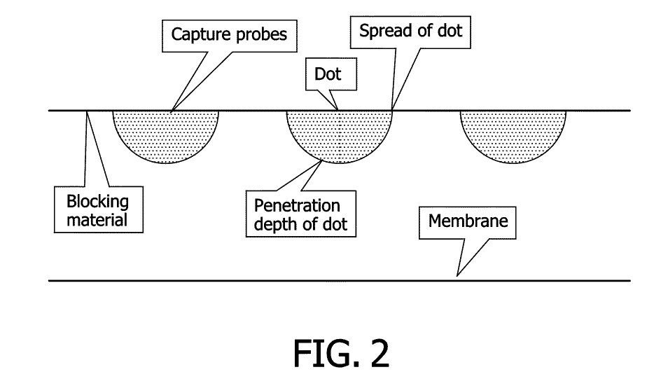

### DETAILED DESCRIPTION OF THE INVENTION

The inventors have found that it is possible to improve several aspects of a deposition approach for a substance when using a transfer solution in order to activate the support onto which the substance is deposited and immobilized.

Although the present invention will be described with respect to particular embodiments, this description is not to be construed in a limiting sense.

Before describing in detail exemplary embodiments of the present invention, definitions important for understanding the present invention are given.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only.

Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

In case the terms “first”, “second”, “third” or “(a)”, “(b)”, “(c)”, “(d)” etc. relate to steps of a method or use there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

As has been set out above, the present invention concerns in one aspect a method for depositing a substance on a support, which comprises (a) providing a substance solution; (b) providing a transfer solution capable of activating the support; (c) depositing said transfer solution on a predefined position of the support; and (d) depositing said substance solution on the same predefined position where the transfer solution was placed, whereby an immobilization of the deposited substance at the location of overlap between the deposited transfer solution and the deposited substance solution on said support is achieved, with the proviso that said transfer solution and said substance solution are not placed together or as a mixed solution on the support.

The term “deposition a substance on a support” relates to the association of a substance to a supportive substrate which positions the substance at a specific area of the supportive substrate. Typically, the specific area of the supportive substrate onto which a substance is to be deposited is a sub-portion of a larger area, preferably comprising between 0.01% and 10%, more preferably comprising between 0.05% and 5% of the entire area of the supportive substrate. A target position may be any reachable point or area on or within a support. Preferably, the term may not include the entire area of a support, e.g. solely refer to a sub-portion thereof. For instance, an entire support area without intervening zones, in which no deposition takes place, may not be comprised by said term.

The term “support” refers to supportive material capable of accepting a charging with substances. The support may be rigid or flexible. The surface of the support may be flat, smooth, rough or porous. Preferably, the support is a solid support. The term “solid support” relates to a material which is mainly of non-liquid consistence and thereby allows for an accurate and trackable positioning of the substance on the support material.

The term “substance” relates to a chemical or biological entity which is amenable to a positioning and immobilization process via the use of a transfer solution. The term “chemical entity” relates to an organic or an organic chemical molecule, e.g. a hydrocarbon, an aliphatic compound, an aromatic compound, a heterocyclic compound, a sugar, a polymer, metal or salt. The term “biological entity” means a biological compound or biomolecule like a protein, a nucleic acid, a lipid, phospholipid or a biological structure like a cell, a cell fragment, a virus, a viral envelope, a cellular membrane or a membrane sub-portion or a biological fluid or liquid like blood, urine, a cell extract, a tissue extract, a tissue exudate, lymph fluid, sputum, saliva or cerebrospinal fluid.

The term “substance solution” relates to a substance as mentioned herein above being comprised in a liquid. Preferably, the term relates to a substance being dissolved in a liquid. The term “liquid” refers to any suitable liquid known to the person skilled in the art, preferably to water based liquids or ionic liquids with a proportion of water in the liquid between 0.1% to 99.9% by volume. The liquid may also comprise further components like a buffer, a salt, or stabilizing agents which prevent the substance from deteriorating, coagulating or precipitating prior to the deposition on the support, as would be known to the person skilled in the art. Examples of such substances are. EDTA, anticoagulants, DNAse inhibitors, RNAse inhibitors, BSA, HSA.

The “substance solution” may have a pH, which is not limited, as long as the substance to be deposited is not modified. Preferably, the solution has a pH ranging from 3 to 12, more preferably from 5 to 10, even more preferably from 6 to 8.5.

Substances may be comprised in a substance solution in an amount of between 0.00000001% and 100% by volume of the substance solution. Preferably, substances may be comprised in a substance solution in an amount of between 0.1% and 80%, more preferably in an amount of 1% to 50%, even more preferably in an amount of between 5% and 35% by volume of the substance solution. High amounts of substances in a substance solution may, for example, be present in cases in which liquid substances are to be deposited. An amount of “100%” means that a pure liquid substance is comprised in the substance solution. Is the substance diluted, e.g. in one or more different liquids, typically in water, the amount by volume may be decreased by the amount of said different liquid or liquids.

Alternatively, substances may be comprised in a substance solution in a concentration of between about 0.001 μM to 100 mM, more preferably of between about 0.01 to 1 mM, and even more preferably of between about 0.1 to 100 μM.

The concentration may vary and/or depend on the nature of the substance, the amount to be deposited, the form and nature of the substrate and other parameters of the depositioning process, which would be known to the person skilled in the art.

The term “transfer solution capable of activating the support” relates to a solution, preferably in liquid form, which may be used in order to facilitate the transfer of a substance to a support. The facilitation of the transfer may be accomplished by an activating reaction on the support. The term “activate” means that the status of the support is changed from non-reactive or inert to reactive with respect to the substance which is transferred to the support. The term “non-reactive” means a state of chemical reactivity or disposition, which can be improved or increased by enhancing means. Preferably, the term denote a state of reactivity which can be enhanced by a factor of about 2 to about 10.000, preferably by a factor of about 5 to about 5000, more preferably by a factor of about 10 to about 1000, even more preferably by a factor of about 15 to about 200 in comparison to a situation in which an activation has been carried out. The activation may be any suitable activation process known to the person skilled in the art, e.g. a chemical, biochemical, mechanical or optical activation. As a result of the activation step, a deposited substance may be immobilized. Alternatively, the activation may prepare the support for a subsequent immobilization upon deposition of a substance. The duration of the activated state of the support is not limited. The activation may have a short duration of milliseconds, seconds or minutes or a longer duration of hours, days, weeks, months or years. The duration of the activated state may depend on the nature, amount and/or form of the deposited substance(s) and/or the activation process and means used, as would be known to the person skilled in the art. Typically, the activation of a support may end when a substance is deposited. Alternatively, in a specific embodiment of the present invention, the activation may be terminated independently of the deposition process, e.g. by using a deactivating or blocking solution. Examples of deactivating or blocking solutions are solutions comprising NaOH (sodium hydroxide) or NH₂-containing groups, e.g. ethylendiamine. Such a deactivating or blocking solution may be deposited simultaneously with a substance solution or, preferably, after the substance solution was deposited. If a deactivating or blocking solution is to be used simultaneously with a substance solution, a deactivating or blocking effect on the activated area may occur after a delay, such that the substance may be efficiently immobilized in the activated areal. The term “delay”, as used herein, denotes a short time interval, which may be due to different reaction velocities.

The term “depositing said transfer solution on a predefined position of the support” means that a transfer solution as mentioned herein above may be placed at a specific area of a supportive material. Typically, the specific area of a supportive material onto which a transfer solution is to be deposited is a sub-portion of a larger area, preferably comprising between 0.01% and 35%, more preferably comprising between 0.05% and 30% and most preferably comprising about 20% of the entire area of the supportive substrate. A target position may be any reachable point or area on or within a support. Preferably, the term may not include the entire area of a support, e.g. solely refer to a sub-portion thereof. For instance, an entire support area without intervening zones, in which no deposition takes place, may not be comprised by said term. The term “predefined position” relates to any reachable point or area on or within a support, which may be selected via suitable means known to the person skilled in the art, e.g. by using appropriate devices or control mechanisms which allow to chose and/or access said reachable points. Examples of such devices are inkjet printing devices, spotting machines etc. Preferably, a predefined position may be located in a distance of between 0.1 μm to 2 cm from a second such position. More preferably, the distance between two such positions is between about 0.5 μm to about 5 mm, even more preferably between about 10 μm to about 2 mm. Most preferred is a distance of 1 mm.

The term “depositing said substance solution on the same predefined position where the transfer solution was placed” as used herein means that a substance solution is placed at the same target position on which a transfer solution was deposited. The term “same target position” denotes the position which has been selected and/or accessed via suitable means known to the person skilled in the art during the deposition of the transfer solution as described herein above. Preferably, the position may comprise a zone of the support material which overlaps in between about 10% to 100% of the area of the deposited transfer solution, preferably in between about 50% to 100%, more preferably in between about 60% to 100%, 80% to 100% or 90% to 100%. Even more preferably, the areas of the deposited transfer solution and the deposited substance solution overlap in between about 95% to 100%.

The term “immobilization of the deposited substance”, as used herein, relates to the durable association of a substance as defined herein above to a supportive substrate, e.g. via molecular interactions which position the substance on the support. The immobilization may prevent a detaching of the substance, e.g. during washing, rinsing or similar liquid interaction steps during the assay. Typically, such molecular interactions are based on the formation of covalent chemical bonds between structural elements or functional groups of the support material and the substance to be immobilized, e.g. corresponding functional groups of the substance to be deposited, as known to the person skilled in the art.

The term “immobilization of the deposited substance at the location of overlap” means that a durable association of a substance as defined herein above to a supportive substrate takes place in areas or zones in which both, a transfer solution and a substance solution has been deposited. The size of the “location of overlap” may be controlled by parameters like the volume of the deposited transfer and/or substance solution, the use of buffer systems which are similar in both, the transfer and the substance solution or environmental parameters like the humidity in the zone of deposition, e.g. in a reaction chamber. Typically, by using identical or almost identical volumes in the transfer and the substance solution, high degrees of overlap may be achieved. The term “almost identical” means that the volume of the transfer solution and the volume of the substance solution may differ by between about 0.0001 to 25%, e.g. by between about 0.0001 to 15%, or by between about 0.0001 to 12%. The volume may differ, for instance, by about 0.1%, 0.2%, 0.3%, 0.4%, 0.5%, 0.6%, 0.7%, 0.8%, 0.9%, 1.0%, 2%, 3%, 4%, 5%, 6%, 7%, 7.5%, 8%, 9%, 10%, 11%, or 12%. The difference may be due to a lower volume of the transfer solution vs. a higher volume of the substance solution or vice versa.

The term “the transfer solution and the substance solution are not placed together on the support”, as used herein, means that the transfer solution and the substance solution as mentioned herein above may not be brought into direct contact and thereby be combined or mingled before the deposition process for either the transfer solution or the substance solution has been terminated. Typically, the substance and the transfer solution may be deposited separately and an immobilization of the deposited substance may only take place in situ, i.e. on the support material.

The term “the transfer solution and the substance solution are not placed as a mixed solution on the support”, as used herein, means that the transfer solution and the substance solution as mentioned herein above may not be combined or mingled before the deposition step on the support material. The term “combined or mingled” denotes a thorough amalgamation of the transfer and substance solution. It is, however, within the scope of the present invention that a transfer and a substance solution as defined herein above may be deposited at the same time, if said transfer and substance solution are present in separable, non-mixed phases as known to the person skilled in the art. Such “non-mixed phases” may be due to the presence of suitable separating structures known to the person skilled in the art, e.g. lipid mono- or bilayers between the transfer solution and the substance solution. For example, the substance solution may be present in micelles, lipid mono- or bilayer-comprising structures or similar structures and the transfer solution may be present in the surrounding liquid environment. Alternatively, the transfer solution may be present in micelles, lipid mono- or bilayer-comprising structures or similar structures and the substance solution may be present in the surrounding liquid environment. Furthermore, both the transfer and the substance solution may be present in micelles, lipid mono- or bilayer-comprising structures or similar structures

In a specific embodiment of the present invention the separation between the transfer and the substance solution may be terminated via, for instance, the local modification of the pH or ion concentration at the location of the deposition or the use of a pore inducing compound or a channel former as known to the person skilled in the art. Examples of such pore inducing compounds are lantibiotics like Nisin, poly-L-lysine, amphotericin B, polymyxins or filipin. Such a pore inducing compound or channel former may be deposited before, during and/or after the deposition of the transfer and/or substance solution. Furthermore, a separated combination of a transfer solution, a substance solution and a pore inducing compound may be placed on a substrate and a mixture or mingling effect of the separated components may be achieved during the deposition process, thereby releasing the pore inducing compound or channel former. The release of the pore inducing compound may be provoked, for example, by shearing forces to a compartment comprising pore inducing compounds during the deposition process. Alternatively, an increase or decrease in the local pH or the ion concentration at the site of deposition may prompt a release of pore inducing compounds, which may subsequently lead to a mixture of the transfer and the substance solution.

In a preferred embodiment of the present invention the interim between the deposition of the transfer solution on a predefined position of the support and the deposition of a substance solution on the same predefined position where the transfer solution was placed may be a predefined fixed period of time. The term “predefined period of time”, as used herein, denotes a period of time between the deposition of the transfer and the substance solution which can be adjusted and settled before the deposition process or during the initiation phase of the deposition process and may be kept during the entire deposition process. The term “deposition process” relates to the deposition of substances on at least one support item, e.g. one physically delimitable piece of support. Alternatively, the term may also refer to the deposition of substances on more than one support item, e.g. a lot or batch of support items, an amount of one day's production number of support items etc.

The period of time between the deposition of the transfer and the substance solution may also be kept for a certain number of deposition actions either during the deposition on one support item or during the deposition on various support items, e.g. a batch of support items, and subsequently be changed and settled at a different value. Such changes or resettlements may be associated, for example, with the employment of a different support type, a different support size, a different deposition method, a different deposition device, modifications in the humidity of the reaction environment, the nature, amount or concentration of the substance to be deposited etc.

The term “fixed period of time” as used herein, denotes a period of time between the deposition of the transfer and the substance solution which may be invariable for more than one individual deposition step. For instance, all deposition steps for the deposition of substance solutions during the depositing process of substances on one support item, e.g. one physically delimitable piece of membrane, may be carried out after an invariable period of time following the deposition of a transfer solution. Alternatively, the deposition of substance solutions on a sub-portion of the processable area of a support item during the depositing process may be carried out in a first invariable period of time after the deposition of a transfer solution, and the period of time between the deposition steps may then be modified to a second invariable period of time for a further sub-portion of the processable area of the support item etc.

The interim between the deposition of a transfer solution in accordance with the present invention and a substance solution in accordance with the present invention may have a duration of milliseconds, seconds, minutes, hours, days, weeks, months or years. Preferably, the interim between the deposition of a transfer solution in accordance with the present invention and a substance solution in accordance with the present invention may have a duration of between about 1 sec to 12 hours, more preferably of between about 10 sec to 1 hour, even more preferably of between about 20 sec to 30 min and most preferably of between about 5 min to 15 min.

In a further preferred embodiment the deposition of the substance solution on the support material is carried out before the deposition of the transfer solution on said support material. The term “before”, as used herein, means that first a substance solution is deposited on a support and subsequently a transfer solution is deposited at the predefined position where the substance solution was placed. Only after the transfer solution is deposited on the support material an immobilization of the deposited substance may be achieved. Typically, when first the substance solution is deposited, no intermediate wetting step like, e.g. a washing or rinsing of the support material may be carried out until after the transfer solution has been deposited. In a specifically preferred embodiment, the application of a transfer solution to the predefined positions where the substance solution was placed may take place at the same time for all deposited substance solution spots. Alternatively, the deposition of the substance solution at all processed positions or a sub-portion of said positions comprising at least two positions of at least one support item may take place after a predefined, fixed period of time, as defined herein above.

The support material in accordance with another preferred embodiment of the present invention may be a material or a substrate comprising functional chemical groups, like amine-reactive groups. The term “amine-reactive group” relates to any chemical group, or biochemical or biological structure which is capable of reacting with amines. Such chemical groups, or biochemical or biological structures are known to the person skilled in the art or may be derived, for example, from chemistry textbooks like Organische Chemie by Hart et al., 2007, Wiley-Vch or Organische Chemie by Vollhardt et al., 2005, Wiley-Vch. The presence and number of functional chemical groups, in particular of amine-reactive groups, on or inside the support material may be controlled and adjusted via suitable chemical modification processes. Such modification processes may, for instance, provide specifically localized functional groups on or within a support material and facilitate a specific interaction between a substance or the substance solution or the transfer solution and the material within the context of these localized functional groups.

The presence and number of functional group on or inside the support material may also have an influence on the orientation and freedom of deposited substances, e.g. deposited macromolecules like nucleic acids etc. For example, the presence of a higher number of functional groups may lead to an immobilization at different points within the deposited substance, e.g. a macromolecule. Furthermore, the presence of corresponding reactive elements within the deposited substance may be used for a control of the orientation of the substance on the support material. Is, for instance, a macromolecule like a nucleic acid to be deposited, an immobilization at the head or tail region or the 5′ or 3′ region of the nucleic acid molecule or an immobilization at the centre region alone or at the centre and the end regions at the same time may be performed.

Furthermore, a specific positioning of functional chemical groups within a support material may be used in order to facilitate a specific interaction between the substance to be deposited and the material within the context of such localized functional groups. Such positioning process may be used, for example, in order to provide an ordered array of deposited substances, e.g. via the use of liquid spotting equipment, preferably ink jet devices. Functional chemical groups or reactive chemical elements on or within the support material may also be masked by a blocking reagent and become available for interaction with substances to be deposited after a de-blocking or de-masking procedure.

In a specific embodiment of the present invention the support comprises carboxylic groups. Accordingly, the term “amine-reactive group” relates to a carboxylic group. The term “carboxylic group” denotes the chemical group CO₂H. This group may be present on chemical, biochemical or biological entities or structures, in particular in carboxylic acids. Its structure is composed of one carbon atom attached to an oxygen atom by a double bond and to a hydroxyl group by a single bond, i.e. a carbonyl group bonded to a hydroxyl group. The carboxyl group has one valence electron in its carbon atom, making it possible to be a part in a larger molecule by bonding through it. Carboxyl groups can only occur at the end of a carbon chain, due to their chemical structure.

A preferred support material is a porous support material or porous substrate. Particularly preferred is nylon, e.g. Nytran N® or Nytran SPC® or Biodyne C®. A further preferred support material or substrate type is a non-porous substrate. Particularly preferred among non-porous substrates are glass, poly-L-lysine coated material, nitrocellulose, polystyrene, cyclic olefin copolymers (COCs), cyclic olefin polymers (COPs), polypropylene, polyethylene and polycarbonate.

Nitrocellulose membranes are the traditional membranes which are generally used fort transfer techniques like Southern blotting. Methods to achieve nucleic acid binding to nitrocellulose, usually by means of physical adsorption, are widely known form the prior art. The principal advantages of nitrocellulose are its ready availability and familiarity. The use of nitrocellulose membranes with radioactive methods of signal detection is well established.

As an alternative to nitrocellulose membranes nylon may be used as a substrate for nucleic acid binding owing to its greater physical strength and binding capacity, and the wider range of available surface chemistries offered, which optimizes, for example, the attachment of substances like nucleic acids. Immobilization on nylon has been demonstrated to be very durable during repeated probe stripping.

The means by which substances, in particular macromolecules, bind to bulk material like, for instance, polystyrene is not well understood. An allocation of binding capacity for bulk materials or its enhancement may be achieved by the provision of functional groups, preferably amine groups, which are made available, e.g. by a coating process or surface treatment or spraying etc. A preferably used coating material is poly-L-lysine, which belongs to the group of cationic surfactants. It contains positively charged hydrophilic (amino) groups and hydrophobic (methylene) groups and is known to interact with nucleic acid molecules.

As bulk material any suitable material known to the person skilled in the art may be used. Typically, glass or polystyrene is used. Polystyrene is a hydrophobic material suitable for binding negatively charged macromolecules because it normally contains few hydrophilic groups.

For macromolecules like nucleic acids, immobilized on glass slides, it is furthermore known that by increasing the hydrophobicity of the glass surface the immobilization of the molecule may be increased. Such an enhancement may permit a relatively more densely packed formation.

In addition to a coating or surface treatment with poly-L-lysine, bulk material, in particular glass, may be treated by silanation, e.g. with epoxy-silane or amino-silane or by silynation or by a treatment with polyacrylamide.

In a further specific embodiment of the present invention bulk material may also be covered with or coated with membrane material as mentioned herein above.

According to a further embodiment of the present invention, the activation of the support conveyed by the transfer solution as defined herein above is a chemical activation. The term “chemical activation”, as used herein, denotes a change of the status of the support from non-reactive to reactive with respect to the substance which is transferred to the support by chemical means. As a result of the chemical activation step, the already deposited substance may be immobilized or the chemical activation may prepare the support for a subsequent immobilization upon deposition of a substance. A chemical activation process may comprise the modification or addition of chemical groups to a substrate which allow a subsequent interaction with deposited substances and/or the enhancement of a reaction of chemical structures present or introduced into a support material with substances deposited on said support.

The term “modification or addition of chemical groups” as used herein denotes the generation of functional chemical groups into a support material, preferably the generation of amine-reactive groups in or on a support material. More preferred is the generation of carboxylic acids on or in a support material. Suitable means and methods for the addition or modification of chemical groups to a support material are known to the person skilled in the art, or can, for example, be derived from chemistry textbooks like Organische Chemie by Hart et al., 2007, Wiley-Vch or Organische Chemie by Vollhardt et al., 2005, Wiley-Vch.

The term “enhancement of a reaction of chemical structures present or introduced into a support material with substances deposited on said support”, as used herein, relates to the increase of yield and/or the decrease of side reactions of a chemical reaction between functional groups, preferably amine-reactive groups, more preferably carboxylic acids, in a support material and corresponding, reactive groups in or on a substance to be deposited and immobilized on the support. The enhancement of a reaction may be an enhancement of the reaction outcome by a factor of about 2 to about 10.000, preferably by a factor of about 5 to about 5000, more preferably by a factor of about 10 to about 1000, even more preferably by a factor of about 15 to about 200 in comparison to a situation in which no chemical activation has been carried out. In a specific embodiment, the enhancement may be an increase of yield by a factor of about 2 to about 10.000, preferably by a factor of about 5 to about 5000, more preferably by a factor of about 10 to about 1000, even more preferably by a factor of about 15 to about 200 in comparison to a situation in which no chemical activation has been carried out. In a further specific embodiment, the enhancement may be a decrease of side reactions by a factor of about 2 to about 10.000, preferably by a factor of about 5 to about 5000, more preferably by a factor of about 10 to about 1000, even more preferably by a factor of about 15 to about 200 in comparison to a situation in which no chemical activation has been carried out. The enhancement may also be combination of an increase of yield and a decrease of side reactions by any of the above mentioned factors.

In a further embodiment of the present invention, the transfer solution capable of activating the support material comprises chemical moieties which are able to react with amine groups or carboxylic groups. Typically, the support material as mentioned herein above comprises carboxylic groups, whereas a substance to be deposited may comprise amine groups. The term “substance comprising amine groups” means that a substance may have a functional amine group or is chemically modified in order to comprise a functional amine group. The term “functional amine group” relates to primary, secondary or tertiary amine groups. The amine group may be either terminal or be comprised in the interior of a substance molecule. Means and methods for a chemical modification in order to generate functional amine groups on or in substances are known to the person skilled in the art and can, for example, be derived from chemistry textbooks like Organische Chemie by Vollhardt et al., 2005, Wiley-Vch.

“Chemical moieties which are able to react with amine groups or carboxylic groups”, as used herein, denotes reactive groups present on compounds or molecules which are capable of conveying a chemical interaction between amine groups and carboxylic groups, preferably of amine groups on one molecule and carboxylic groups on a different molecule, more preferably of amine groups present on a substance to be deposited and immobilized, and carboxylic groups present on a support material. The term also relates to entire compounds or molecules which are capable of conveying a chemical interaction between amine groups and carboxylic groups.

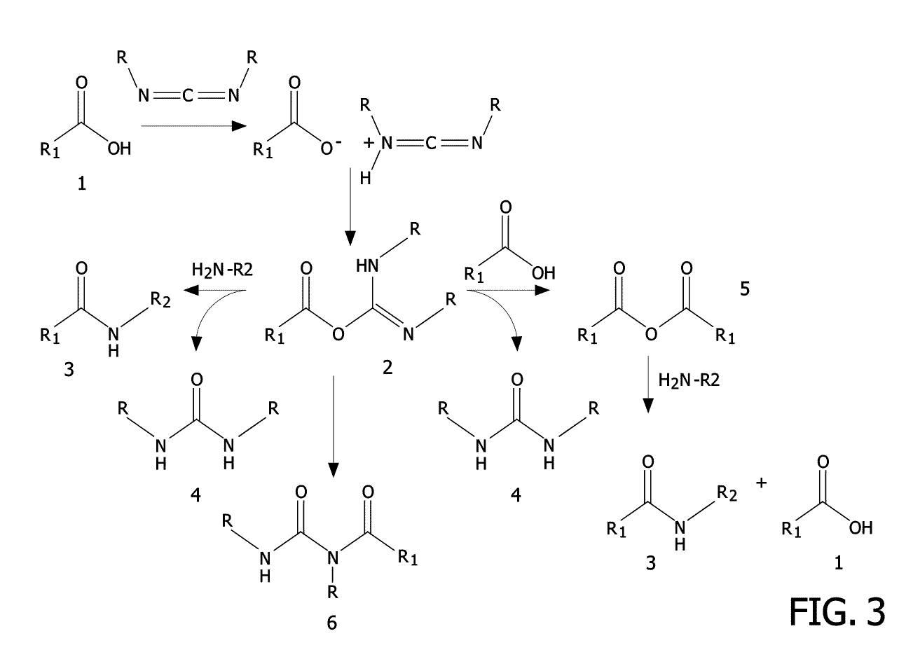

An example of such a chemical moiety is a carbodiimide group or a carbodiimide comprising molecule. A carbodiimide is a functional group or molecule comprising the element N═C═N. Typically, carbodiimides hydrolyze to form ureas. Compounds containing a carbodiimide functionality are dehydration agents and may be used to activate carboxylic acids towards the formation of amides or esters. Typically, the formation of an amide using a carbodiimide comprises the following reaction steps: a carboxylic acid reacts with a carbodiimide to produce key intermediate O-acylisourea, which is a carboxylic ester with an activated leaving group. O-acylisourea will subsequently react with amines to give rise to an amide and urea. A side reaction of O-acylisourea may give rise to different products. For example, O-acylisourea may react with an additional carboxylic acid to produce an acid anhydride, which can produce an additional amide. A further, minor pathway may involve the rearrangement of O-acylisourea to N-acylurea. An illustration of a reaction scheme based on an interaction between a carbodiimide and an amine can be derived from FIG. 3.

Examples of carbodiimides, which may be used in the context of the present invention are N,N′-dicyclohexylcarbodiimide (DCC), N,N′-Diisopropylcarbodiimide (DIC) and 1-ethyl-3-(3-dimethylaminopropyl) carbodiimide hydrochloride (EDC or EDAC).

DCC was one of the first carbodiimides developed. It is widely used for amide and ester formation, especially for solid-phase peptide synthesis. DCC shows a high yielding in amide coupling reactions and is inexpensive.

DIC was developed as an alternative to DCC and is identical to DCC in many ways except that DIC is easier to handle than DCC and that the end-product N,N′-diisopropylurea is soluble in organic solvents and may be removed by extraction.

EDC is a water soluble carbodiimide which may preferably be employed in a pH range of 4.0-6.0. It may be used as a carboxyl activating agent for the coupling of amines, preferably of primary amines, to yield amide bonds. Additionally, EDC may also be used to activate phosphate groups.

A further example of a chemical moiety capable of reacting with amine groups and/or carboxylic groups is N,N′-carbonyl-diimidazole (CDI), which is often used for the coupling of amines, e.g. during the synthesis of peptides.

Another example of a chemical moiety capable of reacting with amine groups and/or carboxylic groups is N-hydroxysuccinimide (NHS). Typically, activated carboxylic acids may react with amines to form amides, whereas a normal carboxylic acid may solely form a salt with an amine. An NHS-activated acid may be synthesized by mixing NHS with a carboxylic acid and a small amount of an organic base, e.g. in an anhydrous solvent. Analogs of NHS, which may also be used in the context of the present invention, are hydroxybenzotriazole (HOBt), 1-Hydroxy-7-azabenzotriazole (HOAt) and pentafluorophenol.

In addition to carbodiimides, CDI or NHS as defined herein above, also derivatives or analogs of these compounds, as known to the person skilled in the art, may be used in the context of the present invention. For example, instead of or together with NHS, water-soluble analogs like N-hydroxysulfosuccinimide (Sulfo-NHS) may be used

In a specific embodiment of the present invention the transfer solution may comprise EDC or NHS or, preferably, a mixture of EDC and NHS. As mentioned herein above, EDC and NHS may be used as activating reagents in order to achieve a reaction between a compound comprising a carboxylic acid group, e.g. present on a substrate material, and a compound comprising an amine group, e.g. present on substance to be deposited and immobilized. A transfer solution according to the present invention may comprise EDC or NHS or both in an appropriate concentration and at a suitable pH, as known to the person skilled in the art.

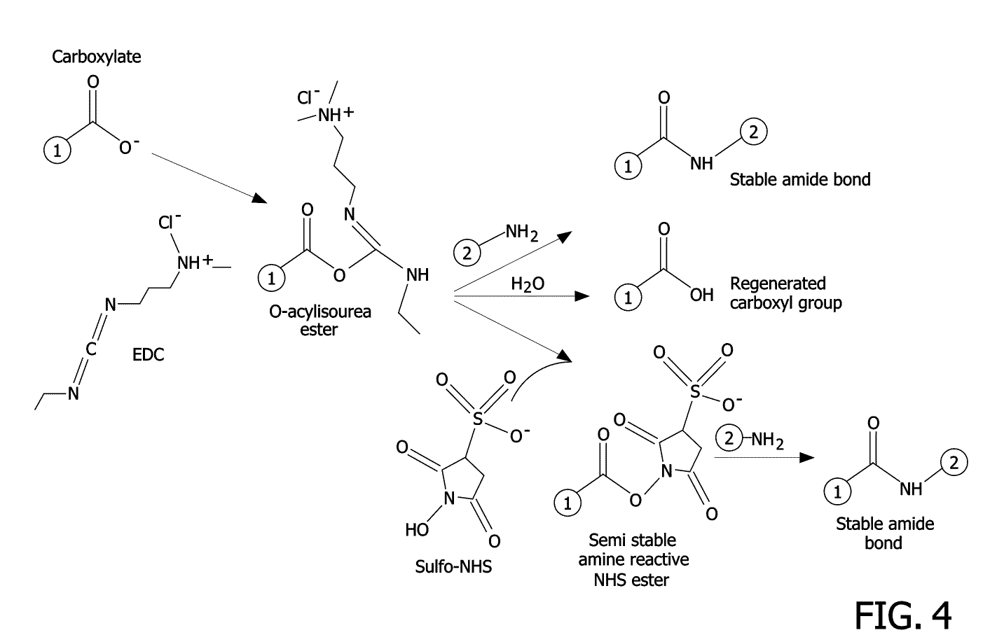

In the presence of Sulfo-NHS, EDC may be used to efficiently convert carboxyl groups to amine-reactive Sulfo-NHS esters, giving yield to stable amides. This may be accomplished by mixing EDC with a carboxyl containing molecule, e.g. present on the support material, and adding Sulfo-NHS. Typically, EDC may react with a carboxyl group, e.g. present on a support material, whereby an amine-reactive O-acylisourea intermediate is formed. This intermediate may react with an amine on a second molecule, e.g. present on a substance according to the present invention, yielding a conjugate of the two molecules joined by a stable amide bond. The intermediate may be susceptible to hydrolysis, making it unstable and short-lived, e.g. in an aqueous solution. The addition of Sulfo-NHS may stabilize the amine-reactive intermediate by converting it to an amine-reactive Sulfo-NHS ester. Thereby the efficiency of the EDC-mediated coupling reaction may be increased. The amine-reactive Sulfo-NHS ester intermediate may have sufficient stability to permit two-step crosslinking procedures, which allows the carboxyl groups on one molecule to remain unaltered. The efficiency of EDC-mediated coupling may accordingly be increased in the presence of Sulfo-NHS. Details of the conversion of carboxyl groups to amides via amine-reactive Sulfo-NHS and EDS may be derived from FIG. 4.

Preferably, Sulfo-NHS may be used in a concentration of between 1 mM to 10 mM, more preferably in a concentration of about 2 mM to 7.5 mM, most preferably in a concentration of 5 mM in a transfer solution according to the present invention. The activation reaction with EDC and Sulfo-NHS may be carried out at any suitable pH in the transfer solution known to the person skilled in the art, preferably at pH 3 to 9, more preferably at pH 4.5 to 7.2. EDC reactions may be carried out in any suitable buffer comprised in the transfer solution known to the person skilled in the art, preferably in MES buffer. EDC reactions may be carried out at any suitable pH in the transfer solution known to the person skilled in the art, preferably at pH 3 to 9, more preferably at pH 4.7 to 6.0. A reaction of Sulfo-NHS-activated molecules with primary amines may preferably be carried out at pH 7 to 8 in the transfer solution.

Alternatively, NHS or any suitable derivative thereof, e.g. Sulfo-NHS, may also be used in combination with other carbodiimides, preferably with one or more of the carbodiimides DCC or DIC as defined herein above.

Furthermore EDC or DCC or DIC may also be used with a NHS analog like, for example, hydroxybenzotriazole (HOBt), 1-Hydroxy-7-azabenzotriazole (HOAt) or pentafluorophenol.

The substance solution may in accordance with a further preferred embodiment of the invention comprise a nucleic acid, a protein or a sugar, or a modified derivative thereof. The substance solution may alternatively comprise any combination of a nucleic acids, proteins, sugars or derivates of any of these. Particularly preferred are nucleic acids, proteins or sugars, or modified derivative thereof which comprise an amine group.

The nucleic acid comprised in the substance solution may be DNA, RNA, PNA, CNA, HNA, LNA or ANA. The DNA may be in the form of, e.g. A-DNA, B-DNA or Z-DNA. The RNA may be in the form of, e.g. p-RNA, i.e. pyranosysl-RNA or structurally modified forms like hairpin RNA or a stem-loop RNA.

The term “PNA” relates to a peptide nucleic acid, i.e. an artificially synthesized polymer similar to DNA or RNA which is used in biological research and medical treatments, but which is not known to occur naturally. The PNA backbone is typically composed of repeating N-(2-aminoethyl)-glycine units linked by peptide bonds. The various purine and pyrimidine bases are linked to the backbone by methylene carbonyl bonds. PNAs are generally depicted like peptides, with the N-terminus at the first (left) position and the C-terminus at the right.

The term “CNA” relates to an aminocyclohexylethane acid nucleic acid. Furthermore, the term relates to a cyclopentane nucleic acid, i.e. a nucleic acid molecule comprising for example 2′-deoxycarbaguanosine.

The term “HNA” relates to hexitol nucleic acids, i.e. DNA analogues which are built up from standard nucleobases and a phosphorylated 1,5-anhydrohexitol backbone.

The term “LNA” relates to locked nucleic acids. Typically, a locked nucleic acid is a modified and thus inaccessible RNA nucleotide. The ribose moiety of an LNA nucleotide may be modified with an extra bridge connecting the 2′ and 4′ carbons. Such a bridge locks the ribose in a 3′-endo structural conformation. The locked ribose conformation enhances base stacking and backbone pre-organization. This may significantly increase the thermal stability, i.e. melting temperature of the oligonucleotide.

The term “ANA” relates to arabinoic nucleic acids or derivatives thereof. A preferred ANA derivative in the context of the present invention is a 2′-deoxy-2′-fluoro-beta-D-arabinonucleoside (2′F-ANA).

The nucleic acid molecules may comprise a combination of any one of DNA, RNA, PNA, CNA, HNA, LNA and ANA. Preferred are mixtures of LNA nucleotides with DNA or RNA bases.

In a preferred embodiment the nucleic acid molecules as defined herein above may be in the form of short oligonucleotides, long oligonucleotides or polynucleotides.

In another embodiment the nucleic acid molecules as defined herein above may be single-stranded or double-stranded. The term “single-stranded nucleic acid” relates to nucleic acid molecules which comprise a single sugar-phosphate backbone and/or are not organized in a helical form. Preferably these nucleic acid molecules exhibit no secondary structures or intermolecular associations. The term “double stranded nucleic acid” relates to nucleic acid molecules which comprise two sugar-phosphate backbones. In a preferred embodiment the double-stranded nucleic acids are organized in a double helical form. In a further embodiment double-stranded nucleic acids according to the present invention may be composed of different types of nucleic acid molecules, e.g. of DNA and RNA, DNA and PNA, DNA and CNA, DNA and HNA, DNA and LNA, DNA and ANA, or RNA and CNA, RNA and PNA, RNA and CNA, RNA and HNA, RNA and LNA, RNA and ANA, or PNA and CNA, PNA and HNA, PNA and LNA, PNA and ANA or CNA and HNA, CNA and LNA, CNA and ANA, or HNA and LNA, HNA and ANA, or LNA and ANA. They may alternatively also be composed of combinations of stretches of any of the above mentioned nucleotide variants.

The nucleic acid comprised in the substance solution which is to be immobilized on the support material may according to a further embodiment of the invention be represented by the formula I:

5′-Yₙ-Xₘ-Bᵣ-Xₚ-Z_{q}-3′

In formula I Y and Z are stretches of nucleotides of only one basetype, wherein Y and Z can be of the same or of a different basetype; X is a spacer; B is a sequence of more than one basetype and n, m, r, p and q are numbers of nucleotides in the nucleic acid, for which the following conditions may apply: n, m, p, q, r>1; n, m, r>1 and p, q=0; p, q, r>1 and n, m=0; n, q, r>1 and m, p=0; n, r>1 and m, p, q=0; q, r>1 and n, m, p=0. The term “stretch of nucleotides of only one basetype” relates to nucleotides composed of only one kind of base, e.g. thymine, guanine, adenine, cytosine or uracil or any functional equivalent derivative thereof. Preferably, the stretches Y and/or Z may be composed of guanine or uracil or thymine.

Y and Z may be present at the same time on the same nucleic acid molecule. In a further embodiment Y and Z may be composed of different basetypes, i.e. Y may be, for example, of basetype uracil, whereas Z may be of basetype guanine or vice versa.

In another embodiment Y and Z may be identical in length or may be different in length. Y and/or Z may have a length of about 2 to about 100 nucleotides, more preferably of about 4 to about 50 nucleotides, even more preferably of about 8 to about 30 nucleotides. Also preferred is a length of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. More preferred is a range of 10-20 nucleotides. Most preferred is a length of 16 nucleotides.

In a format which comprises elements Y and Z at both termini the nucleic acid molecule may comprise in its center a region of specific nucleotides B as depicted herein above in formula I. Alternatively, region B may be connected to only one of Y or Z and thus be located at the terminus of the molecule. The region B may be used for specific detection reactions in a classical hybridisation or microarray approach, i.e. for interaction reactions with oligonucleotides which specifically bind to their complementary region residing within element B. The length and chemical nature of Y and/or Z may have an influence on the flexibility of zone B and, hence, may be used in order to optimize the specific interaction within this zone, e.g. the specific hybridization reactions using complementary oligonucleotides. In a preferred embodiment B has a length of about 4 to about 90 nucleotides, more preferably a length of about 4 to about 50 nucleotides, even more preferably of about 20 to about 30 nucleotides. Preferred lengths are also 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. Most preferred is a length of 25 nucleotides. The stretch of nucleotides of only one basetype as defined herein above may be located at either of both termini of the nucleic acid molecule, i.e. at either the 3′ or the 5′ end of the nucleic acid. More preferably the stretch of nucleotides of only one basetype may be located at the 5′ end of the nucleic acid molecule.

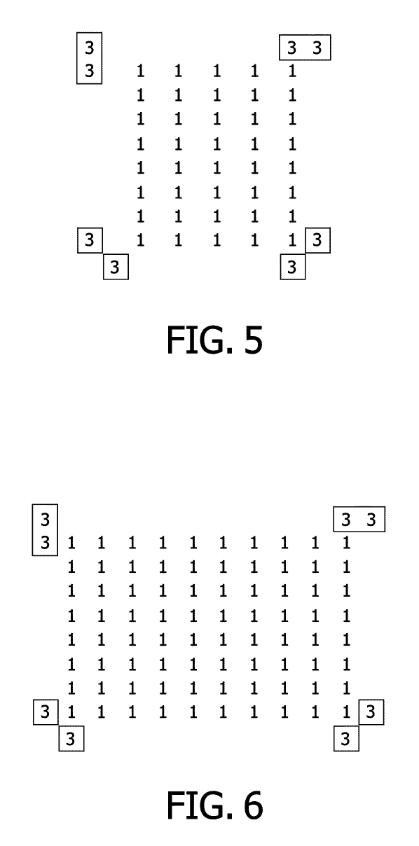

Element(s) X of Formula I of the present invention may additionally be present as spacer element(s), i.e. as regions comprising sequences of undefined nature. More preferably element X may be composed of abasic nucleotides. The term “abasic” relates to positions in the nucleic acid molecule, at which no basic residue is present. Abasic regions or stretches of a nucleic acid are, thus, only composed of sugar phosphate backbone elements. Such an abasic structure may have a positive influence on the flexibility of the entire molecule, in particular with respect to element B of the molecule. The presence of abasic sites has a positive influence on the capability of the immobilized molecule to specifically interact with or hybridize to a target probe (see Example 4 and FIG. 5). A separation of the portions of the molecule used for immobilization, e.g. Y or Z of formula I, form the portion(s) of the molecule used for specific hybridization, e.g. B of formula I, by way of introducing spacer elements comprising abasic sites may significantly decrease unspecific hybridization reactions in the portion of the molecule used for specific hybridization, e.g. B of formula I.

Spacer elements Xm and Xp may entirely be composed of abasic sites or partially be composed of abasic sites. Is the spacer element partially composed of abasic sites the basic portions of the spacer element may be composed of nucleotides of only one basetype or may be composed of nucleotides of different basetypes. Abasic sites as defined herein above may either be accumulated in one stretch or be dispersed within a spacer element or, alternatively, also be present throughout the entire molecule as depicted in formula I. Preferably, the abasic sites are located within the spacer elements X and are accumulated in 1 or 2 stretches.

Preferably, the number of abasic sites within a molecule as depicted in formula I may be between about 1 and about 30, more preferably between about 1 and about 20, even more preferably such a molecule may comprise 1, 2, 3, 4, 5, 6, 7, 8, 9, 10, 11, 12, 13, 14, 15, 16, 17, 18, 19 or 20 abasic sites.

Spacer elements Xm and Xp may be identical in chemical nature and length or may be different in chemical nature and length. Preferably, spacer elements Xm and Xp are of an equal length of about 1 to about 50 nucleotides, more preferably of a length of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nucleotides. In a further embodiment, in case q=0, i.e. no sequence element Z as depicted in formula I is present, also a terminal spacer is avoided, i.e. p=0, Similarly, in case n=0, i.e. no sequence element Y as depicted in formula I is present, also a terminal spacer is avoided, i.e. m=0.

The nucleic acid comprised in the substance solution may according to a further embodiment of the invention comprise one or more labels at either or both of the termini, preferably at the 5′ terminus. Alternatively, said nucleic acid molecules may also comprise one or more labels at any position throughout the molecule. Preferably said nucleic acid molecule comprises between 1 and 10 labels, which may either be identical or different or any combination thereof. More preferably, the nucleic acid molecule or oligonucleotide comprises between 1 and 5 labels, even more preferably 2 labels and most preferably only one label.

Said labels may be radioactive, fluorescent or chemiluminescent labels. The term “radioactive label” relates to labels emitting radioactive radiation, preferably composed of radioactive isotopes. The term “radioactive isotope” in the context of the label relates to any such factor known to the person skilled in the art. More preferably, the term relates to N-15, C-13, P-31 or I-131.

The term “fluorescent label” relates to chemically reactive derivatives of a fluorophore Typically, common reactive groups include amine reactive isothiocyanate derivatives such as FITC and TRITC (derivatives of fluorescein and rhodamine), amine reactive succinimidyl esters such as NHS-fluorescein, and sulfhydryl reactive maleimide activated fluors such as fluorescein-5-maleimide. Reaction of any of these reactive dyes with another molecule results in a stable covalent bond formed between a fluorophore and a labeled molecule. Following a fluorescent labeling reaction, it is often necessary to remove any nonreacted fluorophore from the labeled target molecule. This may be accomplished by size exclusion chromatography, taking advantage of the size difference between fluorophore and labeled nucleic acid or oligonucleotide. Fluorophores may interact with the separation matrix and reduce the efficiency of separation. For this reason, specialized dye removal columns that account for the hydrophobic properties of fluorescent dyes may be used. A particular advantage of fluorescent labels is that signals from fluorescent labels do not disperse. The lack of dispersal in the fluorescent signal permits, for example, a denser spacing of probes on a support. Another advantage of fluorescent probes is that an easy multiple-color hybridization detection may be carried out, which permits direct quantitative determination of the relative abundance of oligonucleotides forming a complex with the nucleic acid molecules immobilized on the support material. In a particularly preferred embodiment the fluorescent labels FITC, Fluorescein, Fluorescein-5-EX, 5-SFX, Rhodamine Green-X, BodipyFL-X, Cy2, Cy2-OSu, Fluor X, 5 (6) TAMRA-X, Bodipy TMR-X, Rhodamine, Rhodamine Red-X, Texas Red, Texas Red-X, Bodipy TR-X Cy3-OSu, Cy3.5-OSu, Cy5, Cy5-Osu, Alexa fluors, Dylight fluors and/or Cy5.5-OSu may be used. These labels may be used either individually or in groups in any combination.

The term “chemiluminescent lable” relates to a label which is capable of emitting light (luminescence) with a limited emission of heat as the result of a chemical reaction. Preferably, the term relates to luminol, cyalume, oxalyl chloride, TMAE (tetrakis (dimethylamino) ethylene), pyragallol, lucigenin, acridinumester or dioxetane.

The immobilization of a nucleic acid molecule to the support material may in accordance with a further preferred embodiment of the invention be based on a coupling between an amine-modified nucleic acid and an element of the support material comprising a corresponding functionality, i.e. a functional chemical group which predominantly interacts with amine-modified nucleic acid molecules, e.g. a carboxylic group as defined herein above. More preferably, the interaction between a nucleic acid comprising at least one amine group and a support material comprising carboxylic groups may be enhanced by the presence of carbodiimides, e.g. EDS, DIC or DCC and/or the presence of NHS or analogs thereof, as described herein above.

The term “amine modified” relates to the introduction, activation or modification of amine groups within the nucleic acid molecule with the purpose of establishing reactive functional amine groups. Such amine groups may, for example, be introduced throughout the length of the molecule. Preferably the groups are introduced at both or one of the termini of the molecule or at its center. Such a modification may be used in order to control and shape the binding behavior of the molecule on the support.

A protein comprised in the substance solution may be any protein, polypeptide or peptide, preferably any protein, polypeptide or peptide up to a size of 1000 kDa. In a preferred embodiment the protein may comprise at least one amine group, which may be located either terminally or internally. Such amine groups may, for example, be introduced throughout the length of the molecule. Preferably the groups are introduced at both or one of the termini of the molecule or at its center. Such a modification may be used in order to control and shape the binding behavior of the molecule on the support. The immobilization of a protein molecule to the support material may in accordance with a further preferred embodiment of the invention be based on a coupling between an amine-modified or amine-comprising protein and an element of the support material comprising a corresponding functionality, i.e. a functional chemical group which predominantly interacts with amine-modified or amine comprising protein molecules, e.g. a carboxylic group as defined herein above. More preferably, the interaction between a protein comprising at least one amine group and a support material comprising carboxylic groups may be enhanced by the presence of carbodiimides, e.g. EDS, DIC or DCC and/or the presence of NHS or analogs thereof, as described herein above.

A protein comprised in the substance solution in accordance with the present invention may be a purified protein or a newly synthesized protein. The term “purified” relates to purification processes known to the person skilled in the art, based, e.g., on the use of gel filtration, affinity chromatography, ion exchange chromatography etc. A purified protein may comprise minor residuals of cell debris, culture supernatant or buffers etc.

A sugar comprised in the substance solution may be any sugar known to the person skilled in the art, e.g. derivable from a biochemistry textbook like Biochemistry, 2006, Berg, Tymoczko and Stryer, Palgrave Macmillan, 6^{th} edition. Typically, the sugar may be a monosaccharide, a disaccharide, a trisaccharide, an oligosaccharide or a polysaccharide. A monosaccharide may, in the context of the present invention, be a trioses, e.g. a ketotriose like dihydroxyaceton or an aldotriose like glyceraldehydes, a tetrose, e.g. a ketotetrose like erythrulose or an aldotetroses like erythrose or threose, a pentose, e.g. a ketopentose like ribulose or xylulose, an aldopentose like ribose, arabinose, xylose, lyxose, a deoxy sugar like deoxyribose, or a hexose, e.g. a ketohexose like psicose, fructose, sorbose, or tagatose, or a aldohexose like allose, altrose, glucose, mannose, gulose, idose, galactose, talose, a deoxy sugar like fucose, fuculose or rhamnose or a heptose like sedoheptulose. A disaccharide may, in the context of the present invention, be a sucrose, lactose, trehalose, or maltose. A trisaccharide may, in the context of the present invention, be a raffinose, melezitose, or maltotriose. A tetrasaccharides may, in the context of the present invention, be an acarbose or a stachyose. An oligosaccharide may, in the context of the present invention, be a fructooligosaccharide (FOS), a galacto-oligosaccharide (GOS) or a mannan-oligosaccharides (MOS). A polysaccharide may, in the context of the present invention, be glycogen, starch (amylase or amylopectin), cellulose, dextrin, glucan (e.g. geta-glucan), fructan (e.g. inulin, levan beta 2→6) or chitin.

In a preferred embodiment the sugar may comprise at least one amine group, which may be located either internally or, in particular in the case of oligo- and polysaccharides, be located terminally. Such amine groups may, for example, be introduced throughout the length of the molecule. Preferably the groups are introduced at both or one of the termini of the molecule or at its center. Such a modification may be used in order to control and shape the binding behavior of the molecule on the support. The immobilization of a sugar molecule to the support material may in accordance with a further preferred embodiment of the invention be based on a coupling between an amine-modified or amine-comprising sugar molecule and an element of the support material comprising a corresponding functionality, i.e. a functional chemical group which predominantly interacts with amine-modified or amine comprising sugar molecules, e.g. a carboxylic group as defined herein above. More preferably, the interaction between a sugar molecule comprising at least one amine group and a support material comprising carboxylic groups may be enhanced by the presence of carbodiimides, e.g. EDS, DIC or DCC and/or the presence of NHS or analogs thereof, as described herein above.

In another embodiment of the present invention, the chemical entity or molecule comprised in the substance solution may be an abietic acid, acenaphthene, acenaphthoquinone, acenaphthylene, acetaldehyde, acetamide, acetaminophen, acetaminosalol, acetamiprid, acetanilide, acetic acid, acetoguanamine, acetone, acetonitrile, acetophenone, acetylcholine, acetylene, N-acetylglutamate, acetylsalicylic acid, fuchsin, acridine, acridine orange, acrolein, acrylamide, acrylic acid, acrylonitrile, acryloyl chloride, adamantane, adenosine, adipamide, adipic acid, adiponitrile, adipoyl dichloride, adonitol, adrenochrome, aflatoxin, alanine, aldosterone, aldrin, alizarin, allantoic acid, allantoin, allethrin, allyl propyl disulfide, allylamine, allyl chloride, p-aminobenzoic acid (PABA), aminodiacetic acid, aminoethylpiperazine, 5-amino-2-hydroxybenzoic acid, aminophylline, 5-aminosalicylic acid, aminothiazole, amiodarone, amiton, amyl nitrate, amyl nitrite, anethole, angelic acid, anilazine, aniline, aniline hydrochloride, anisole, anisoyl chloride, anthanthrene, anthracene, anthramine, anthranilic acid, anthraquinone, anthrone, antipyrine, aprotinin, arabinose, arginine, aroclor, ascorbic acid (vitamin C), asparagine, asparagusic acid, aspartame, aspartic acid, asphidophytidine, atrazine, aureine, avobenzone, azadirachtin, azathioprine, azelaic acid, aziridine, azithromycin, azobenzene, azulene, behenic acid, benomyl, benzaldehyde, benzalkonium chloride, benzamide, benzanthrone, benzene, benzethonium chloride, benzidine, benzil, benzilic acid, benzimidazole, benzisothiazolinone, benzisoxazole, benzo(a)anthracene, benzo(c)cinnoline, benzo(a)pyrene, benzo(c)phenanthrene, benzo(e)fluoranthene, benzo(e)pyrene, benzo(ghi)perylene, benzo(j)fluoranthene, benzo(k)fluoranthene, benzo(c)thiophene, benzocaine, benzofuran, benzoic acid, benzoin, benzothiazole, benzothiophene, benzotriazole, benzoxazole, benzoyl chloride, benzyl alcohol, benzyl chloroformate, benzylamine, benzyldimethylamine, benzylidene acetone, betaine, betulin, butylated hydroxytoluene, biotin (vitamin H), biphenyl, 2,2′-bipyridyl, 1,8-bis(dimethylamino)naphthalene, bis(chloromethyl)ether, bisphenol A, biuret, borneol, brassinolide, bromacil, bromoacetic acid, bromobenzene, 2-bromo-1-chloropropane, bromocyclohexane, bromoform, bromomethane, 2-bromopropane, bromotrifluoromethane, brucine, buckminsterfullerene, buspirone, 1,3-butadiene, butadiene resin, butane, butene, 2-butoxyethanol, butylamine, butyllithium, 2-butyne-1,4-diol, butyraldehyde, butyrophenone, butyryl chloride, cacodylic acid, cacotheline, cadaverine, cadinene, cafestol, caffeine, calcein, calciferol, calcitonin, calmodulin, calreticulin, camphene, camphor, cannabinol, caprolactam, caprolactone, capsaicin, captan, captopril, carbazole, carbofuran, carbonyl fluoride, carboplatin, carboxypolymethylene, carminic acid, carnitine, carvacrol, carvone, catechol, cefazolin, cefotaxime, ceftriaxone, cellulose, cellulose acetate, cetrimide, cetyl alcohol, chloracetyl chloride, chloral, chloral hydrate, chlorambucil, chloramine-t, chloramphenicol, chloranilic acid, chlordane, chlorhexidine gluconate, chloro-m-cresol, chloroacetic acid, 4-chloroaniline (p-chloroaniline), chlorobenzene, 2-chlorobenzoic acid (o-chlorobenzoic acid), chlorodifluoromethane, chloroethene, chlorofluoromethane, chloromethane, 2-chloro-2-methylpropane, chloronitroaniline, chloropentafluoroethane, chloropicrin, chloroprene, chloroquine, chlorostyrene, chlorothiazide, chlorotrifluoromethane, chlorotrimethylsilane, chloroxuron, chlorpyrifos, chlorthiamide, cholesterol, choline, chromotropic acid, cilostazol, cinchonine, cinnamaldehyde, cinnamic acid, cinnamyl alcohol, cinnoline, cis-2-butene, cis-3-hexenal, cis-3-hexen-1-ol, citral, citric acid, citrulline, clobetasone, clopidol, cobalamin (vitamin B12), cocamidopropyl, colchicine, collagen, collodion, coniine, coronene, coumarin, creatine, cresol, crotonaldehyde, cubane, cumene, cupferron, cuscohygrine, cyanogen, cyanogen chloride, cyanoguanidine, cyanuric acid, cyanuric chloride, cyclodecane, α-cyclodextrin, cyclododecane, cycloheptatriene, 1,3-cyclohexadiene, 1,4-cyclohexadiene, cyclohexane, cyclohexanol, cyclohexanone, cyclohexene, cyclonite, cyclooctatetraene, cyclopentadiene, cyclopentane, cyclopentanol, cyclopentanone, cyclopentene, cypermethrin, cysteamine, cysteine, decaborane, decabromodiphenyl ether, decahydronaphthalene, decane, dehydroacetic acid, dehydrocholic acid, deltamethrin, dexamethazone, dextran, dextrin, 3,3′-diaminobenzidine, di-t-butyl peroxide, diacetylene, diazinon, diazomethane, 1,2-dibromoethane, dibucaine hydrochloride, dichloroacetic acid, p-dichlorobenzene, dichlorobutane, dichlorodifluoromethane, dichlorodimethylsilane, 1,2-dichloroethane, dichlorofluoromethane, dichlorophen, 2,4-dichlorophenoxyacetic acid, dichlorotrifluoroethane, dicofol, dicyclopentadiene, dieldrin, diethanolamine, diethion, diethyl aluminium chloride, diethylamine, diethylene glycol, diethylenetriamine, diethyl ether, difluoromethane, digitonin, dihydrocortisone, diisoheptyl phthalate, diisopropyl ether, diketene, dimethicone, dimethylamine, N,N-dimethylacetamide, N,N-dimethylaniline, 1,2-dimethylbenzene (o-xylene), 1,3-dimethylbenzene (m-xylene), 1,4-dimethylbenzene (p-xylene), N,N-dimethylformamide, dimethyldiethoxysilane, dimethylglyoxime, dimethylmercury, dimethyl sulfoxide, dioctyl phthalate, dioxane, dioxathion, dioxin, diphenylacetylene, diphenylmethanol, disulfuram, disulfoton, dithranol, 2,6-di-tert-butylphenol, 2,6-di-tert-butyl-4-methylphenol, 2,6-di-tert-butylpyridine, diuron, divinylbenzene, docosane, dodecane, dodecylbenzene, dopamine, doxylamine succinate, eicosane, endosulfan, endrin, eosin, ephedrine, epibromohydrin, epinephrine, erucic acid, erythritol, estradiol, ethacridine lactate, ethane 1,2-ethanedithiol, ethanol, ethene, ethidium bromide, ethyl acetate, ethylamine, ethyl 4-aminobenzoate, ethylbenzene, ethyl chloride, ethylene, ethylene glycol, ethylene oxide, ethyl formate, 2-ethyl-1-hexanol, eugenol, farnesol, ferrocene, flunixin, fluoranthene, fluorene, 9-fluorenone, fluorescein, fluorobenzene, fluoroethylene, fluoxetine, folic acid (vitamin M), formaldehyde, formamide, formanilide, formic acid, formoterol, fumaric acid, furan (furane), furfural, furfuryl alcohol, furfurylamine, furylfuramide, gadopentetate, galactose, gamma-aminobutyric acid, gamma-butyrolactone, gamma-hydroxybutyrate, geraniol, gibberellic acid, gluconic acid, glutamic acid, glutamine, glutaraldehyde, glutaric acid, glutathione, glyburide, glycerol, glycerophosphoric acid, glycidol, glycine, glycogen, glycolic acid, glyoxal, guanidine, guanine, guanosine, halothane, hematoxylin, heptadecane, heptane, hexabromocyclododecane, hexachloropropene, hexadecane, hexafluoro-2-propanol, hexafluoro-2-propanone, hexafluoroethane, hexafluoropropylene, hexamethyldewarbenzene, hexamethyldisilazane, hexamethylenimine, hexamethylolmelamine, hexamine, hexane, hexanitrodiphenylamine, hexanoic acid, cis-3-hexanal, cis-3-hexen-1-ol, hippuric acid, histidine, histamine, homoarginine, homocysteine, homocystine, homotaurine, hydrochlorothiazide, hydroquinone, hydroxyproline, 5-hydroxytryptamine, imidazole, indazole, indene, indole, indoline, indole-3-acetic acid, inositol, iodoxybenzene, isatin, isoamyl isobutyrate, isobenzofuran, isoborneol, isobornyl acetate, isoflurane, isoindole, isoleucine, isomelamine, isooctanol, isophthalic acid, isopropanol, isoquinoline, isoxazole, jasmone, keratin, ketene, kojic acid, lactic acid, lactose, lauric acid, lauryl alcohol, lithium diisopropylamide, leucine, levulinic acid, limonene, linalool, linoleic acid, linolenic acid, lipoamide, lithium diisopropylamide, loratadine, luminol, 2,6-lutidine, lycopene, lysine, malathion, maleic anhydride, malic acid, maltose, mandelonitrile, mannide monooleate, mannose, melatonin, menthol, 2-mercaptoethanol, 2-mercaptopyridine, merocyanine, mesityl oxide, mesitylene, mesotartaric acid, metaldehyde, metamizole, methanesulfonic acid, methanol, methionine, methomyl, 4-methoxybenzaldehyde, methoxychlor, methoxyflurane, methyl acetate, methyl-2-cyanoacrylate, methyl ethyl ketone, methyl isobutyl ketone, methyl isocyanate, methyl methacrylate, methyl tert-butyl ether, methylal, methylamine, 2-methylbenzoic acid, 4-methylbenzoic acid, methyl chloroformate, methylcyclohexane, methylhydrazine, methylmorpholine, 2-methylpropene, N-methylpyrrolidone, methyltriethoxysilane, methyltrimethoxysilane, metoprolol, metronidazole, milrinone, monocrotophos, monosodium glutamate, myrcene, N-nonadecane, N-tetradecylbenzene, naphthalene, naphthoquinone (vitamin K), 2-naphthylamine, niacin (vitamin B3), nicotine, niflumic acid, nimesulide, nitrilotriacetic acid, nitrobenzene, nitroethane, nitrofen, nitrofurantoin, nitromethane, nitrosobenzene, N-nitroso-N-methylurea, nitrosomethylurethane, nominine, nonacosane, nonane, noradrenaline, norepinephrine, norephidrine, norcarane, norleucine, nujol, octabromodiphenyl ether, octane, 1-octanethiol, octanoic acid, 4-octylphenol, oleic acid, orcin, orcinol, ornithine, orotic acid, oxalic acid, oxalyl chloride, oxamide, oxazole, oxolinic acid, oxymetholone, p-nitro benzal dehyde, paba, palmitic acid, pantothenic acid (vitamin B5), parachlorometaxylenol, paraformaldehyde, parathion, pelargonic acid, pentabromodiphenyl ether, pentachlorobiphenyl, pentachlorophenol, pentadecane, pentaerythritol, pentaethylene glycol, pentafluoroethane, pentane, pentetic acid, perfluorotributylamine, permethrin, peroxyacetic acid, perylene, phenacetin, phenacyl bromide, phenanthrene, phenanthrenequinone, phencyclidine, phenethylaminephenol, phenolphthalein, phenothiazine, phenylacetic acid, phenylacetylene, phenylalanine, p-phenylenediamine, phenylhydrazine, phenylhydroxylamine, phenyllithium, 4-phenyl-4-(1-piperidinyl)cyclohexanol, phenylthiocarbamide, phloroglucinol, phorate, phthalic anhydride, phthalic acid, phytic acid, 4-picoline, picric acid, pimelic acid, pinacol, piperazine, piperidine, piperonal, piperylene, pivaloyl chloride, polyacrylonitrile, polybenzimidazole, polyethylenimine, polygeline, polyisobutylene, polypropylene, polypropylene glycol, polystyrene, polyurethane, polyvinyl acetate, polyvinyl alcohol, polyvinyl chloride, polyvinylidene chloride, polyvinylidene fluoride, polyvinylpyrrolidone, porphyrin, potassium clavulanate, potassium 2-ethyl hexanoate, prednisone, primaquine, progesterone, prolactin, proline, propanoic acid, 2-propanone, propargyl alcohol, propiconazole, propiolactone, propiolic acid, propionaldehyde, propionitrile, propoxur, purine, putrescine, pyrazine, pyrazole, pyrene, pyrethrin, pyridazine, pyridine, pyridinium tribromide, 2-pyridone, pyridoxal, pyridoxine (vitamin B₆), pyrilamine, pyrimethamine, pyrimidine, pyroglutamic acid, pyrrole, pyrrolidine, pyruvic acid, quinaldine, quinazoline, quinhydrone, quinoline, quinone, quinoxaline, raffinose, resorcinol, retinene, retinol (vitamin A), rhodanine, riboflavin (vitamin B2), ribofuranose, ricin, rosolic acid, rotane, rotenone, saccharin, safrole, salicin, salicylaldehyde, salicylic acid, salvinorin A, sclareol, sebacic acid, sebacoyl chloride, selacholeic acid, selenocysteine, selenomethionine, serine, serotonin, shikimic acid, skatole, sorbic acid, spermidine, squalene, stearic acid, styrene, succinic anhydride, sulfanilamide, sulfanilic acid, sulforhodamine B, suxamethonium chloride, tannic acid, tannin, tartaric acid, tartrazine, taurine, terephthalic acid, terephthalonitrile, p-terphenyl, α-terpineol, testosterone, tetrachlorobiphenyl, tetrachloroethylene, tetrachloromethane, tetradecane, tetraethylene glycol, tetrafluoroethene, tetrahedrane, tetrahydrofuran, tetrahydronaphthalene, tetramethrin, tetramethylsilane, tetramethylurea, tetranitromethane, tetrathiafulvalene, tetrazine, tetrodotoxin, thiamine (vitamin B1), thiazole, thioacetamide, thiolactic acid, thiophene, thiophosgene, thiourea, thiram, thorin, threonine, thrombopoietin, thymidine, thymine, thymol, thymolphthalein, thyroxine, tiglic acid, timidazole, tocopherol (vitamin E), toluene, toluene diisocyanate, p-toluenesulfonic acid, o-toluic acid, p-toluic acid, toxaphene, triangulane, triazole, tributyl phosphate, tributylamine, tributylphosphine, trichloroacetic acid, trichloroacetonitrile, 1,1,1-trichloroethane, trichloroethylene, trichlorofluoromethane, 2,4,6-trichloroanisole, 2,4,6-trichlorophenol, tricine, triclabendazole, tridecane, tridecanoic acid, triethylaluminium, triethylamine, triethylamine hydrochloride, triethylene glycol, triethylenediamine, trifluoroacetic acid, 1,1,1-trifluoroethane, 2,2,2-trifluoroethanol, trifluoromethane, trimellitic anhydride, trimethoxyamphetamine, trimethyl phosphite, trimethylamine, trimethylbenzene, 2,2,4-trimethylpentane, tri-o-cresyl phosphate, triphenyl phosphate, triphenylamine, triphenylene, triphenylmethane, triphenylmethanol, triphenylphosphine, tropane, tropinone, tryptophan, tyrosine, umbelliferone, undecanol, uracil, urea, urethane, uric acid, uridine, usnic acid, valine, vanillin, venlafaxine, vinyl acetate, vinyl fluoride, vinylidene chloride, warfarin, xanthone, xylene, xylose, yohimbine hydrochloride, yohimbinic acid monohydrate or zingiberene, or any derivative thereof, or any combination of any of the above mentioned compounds. Any of these chemical entities or molecules may be present in a liquid, preferably in a suitable buffer and/or at a suitable pH, as known to the person skilled in the art. The chemical entities or molecules may comprise or be linked to functionalized groups, e.g., amine groups, in order to be capable of being immobilized on a support material. Preferably, the immobilization may take place between an amine-reactive functionality on the support material, e.g. carboxylic groups, and amine groups present in the substance to be deposited. Typically, the immobilization process may be enhanced by the presence of carbodiimide groups and/or enhancer molecules like NHS. Preferably, the presence of EDC and NHS may be used in order to enhance the interaction between amine groups on chemical substance molecules and carboxylic groups in or on support material.

According to another preferred embodiment of the present invention, subsequent to the immobilization of the deposited substance at a location of overlap between the deposited transfer solution and the deposited substance solution, in a further step (e) the support may be washed or rinsed. By washing or rinsing the support material, substance solution, which is not fixated according to a previous immobilization step, or residual transfer solution or any other items not immobilized on the support material may be removed. Preferably, washing or rinsing steps may be carried out with an appropriate washing or rising buffer, as known to the person skilled in the art. A washing or rinsing buffer to be used in the context of the present invention typically comprises salts. Typical salts which may be used in washing buffers are SSC, SSPE or PBS. Furthermore, the buffer may comprise additional ingredients such as detergents like SDS (preferably between 0.01-0.5%), or Tween 20. Moreover, the buffer may comprise bulk DNA, like herring sperm DNA (hsDNA), or blocking agents like BSA. For example, a washing buffer may comprise 2×SSC and 0.05% SDS (solution 1), or 0.1×SSC and 0.1% SDS (solution 2). Alternatively, the washing buffer may comprise 2×SSC, 10 mM Tris-HCl pH7.5 and 0.5% SDS (solution 1), or 1×SSC, 10 mM Tris-HCl pH7.5 and 0.5% SDS (solution 2). Solution 1 and 2, as defined herein above, may be used together, preferably solution 1 is used first and solution 2 is used afterwards. The washing or rinsing may be carried out for a predefined period of time, e.g. for between about 10 to 60 minutes, preferably for 15 minutes. The washing or rinsing step may be repeated various times, preferably it may be repeated once or twice. The washing or rising step repetitions may differ in terms of amount of time used.

Furthermore, the washing or rinsing procedure may be carried out at any suitable temperature known to the person skilled in the art. Preferably, the washing or rising step may be carried out at room temperature or in a temperature range of between about 35° C. to 60° C. Preferably, the washing or rinsing step may be carried out at a temperature of 55° C. Temperature ranges or temperatures may be changed for repetitions of the washing or rinsing step. Preferably, a first washing step may be carried out at room temperature, followed by a second washing step carried out at 55° C.

Further particulars, such as alternative buffers, temperature ranges, pH, ingredients etc., which may also be used in the context of the present invention, are known to the person skilled in the art and can be derived from, e.g., Sambrook et al., Molecular Cloning: A Laboratory Manual, 2001, Cold Spring Harbor Laboratory Press.

The washing or rinsing may preferably be performed after a certain period of time subsequent to the termination of the deposition and immobilization process. Typically, a period of at least between about 0.2 and 30 minutes, preferably a period of between about 5 to 10 minutes may elapse after the termination of the of the deposition and immobilization process before a washing or rinsing procedure may be started.

Additionally or alternatively, the support material may be dried. A drying step may enhance the deposition and immobilization efficiency. The term “drying”, as used herein, denotes a storing at room temperature or any other suitable temperature, e.g. an elevated temperature up to 70° C. or an active drying process based on the use of an air flow, preferably the flow of hot air, having e.g. an elevated temperature of up to 70° C. Alternatively, the drying may be performed by using dry nitrogen, e.g. for time period of between about 2 sec to 5 min, preferably for a time period of between about 5 sec to 60 sec. For the application of the nitrogen during the drying step any suitable means known to the person skilled in the art may be used. Typically, a nitrogen pistol may be used.

A drying step may be carried out between the deposition of the transfer solution and the deposition of the substance solution or vice versa. Typically, a drying step, as defined herein above, may be performed for a time period of between about 1 min to 30 min, more preferably for a time period of between about 2 min to 10 min, even more preferably for a time period of about 5 min. The effect of the drying process may be assessed with any suitable means known to the person skilled in the art, e.g. with optical detector systems, CCD cameras, hygrometers etc. In a preferred embodiment of the present invention, the substance solution may only be deposited when the spot, where the transfer solution has been placed, is relatively dry. Alternatively, in a further embodiment of the present invention, the transfer solution may only be deposited when the spot, where the substance solution has been placed, is relatively dry. Such a state may be checked and verified with any suitable assessment methods, e.g. those defined herein above. The term “relatively dry”, as used herein, means that the amount of liquid, e.g. vaporable liquid, in the spot of deposited solution is decreased by at least about 65%, preferably by at least about 75%, more preferably by at least about 85% and most preferably by at least about 95-99% in comparison to the amount of liquid present in the spot in the moment of, or directly after the deposition.

In another aspect, the present invention relates to the use of a method for depositing a substance on a support as defined herein above for the manufacturing of a chip.

In yet another aspect, the present invention relates a method for manufacturing a chip as defined herein above, wherein the substance is deposited on a chip substrate according to any of the methods for depositing a substance on a support as defined herein above.

Furthermore, in an additional aspect, the present invention relates to a chip manufactured according a method for manufacturing a chip as defined herein above.

The term “chip”, as used herein, denotes a collection of miniaturized test sites arranged on a support, produced in accordance with the methods of the present invention as defined herein above, which permits assays or tests to be performed. Such an arrangement typically permits to save time and to achieve a high output and speed during assay, assessment or test processes. Typically, a chip comprises a support material which may be either open or packaged. If the chip is packaged, it may comprise, in addition to the support material a reaction chamber or cavity comprising the support material, preferably formed between a first surface and a second surface, wherein the second surface is located opposite to the first surface. The term “reaction chamber” denotes the space formed within a chamber body between a first surface and a second surface. The reaction chamber may be laterally limited by sidewalls. The second surface may be located opposite to the first surface. Preferably, the first surface and the second surface may be arranged in parallel or substantially parallel to each other.

The term “manufacturing of a chip” as used herein relates to the use of a method for depositing a substance on a support as defined herein above for the fabrication of a support material comprising the deposited substances in an immobilized form. The produced support material, i.e. the basic chip may be packaged in the form of a device or reaction chamber system or may be used as such. Support material to be used for the manufacturing of a chip may be selected from a wide range of material as has been defined herein above. The support material to be used for the manufacturing of a chip may exist as particles, strands, precipitates, gels, sheets, tubing, spheres, containers, capillaries, pads, slices, films, plates or slides, etc. The support material for the manufacturing of a chip according to the present invention may have any convenient shape known to the person skilled in the art, such as a disc, square, sphere, circle, etc. The support material may preferably be flat but may take on a variety of alternative surface configurations. For example, the support material may contain raised or depressed regions on which a substance may be located. The support material and its surface may preferably be rigid. The support material and its surface may alternatively be chosen to provide appropriate light-absorbing characteristics, as would be known to the person skilled in the art.

In case a packaged chip is to be produced or manufactured, the surface, board or packaging barriers or elements, e.g. the first surface and the second surface, may be made of the same material or of different materials. It is also possible that the first surface and/or the second surface comprise(s) surface areas made of different materials, for example, one surface area is made of a transparent material, whereas the remaining surface area is made of a non-transparent material. The first surface and/or the second surface may, for example, comprise a central, optionally rectangular, surface area made of transparent material, whereas the remainder of the surface area (i.e. the “border”) may be made of a non-transparent material.

In preferred embodiments of the invention, at least a part of the surface, board or packaging barriers, in particular of the first surface and/or the second surface may be made of an amorphous material. The term “amorphous material”, as used herein, refers to a solid in which there is no long-range order of the positions of the atoms, i.e. a non-crystalline material. Examples of such amorphous materials include inter alia ceramic materials such as aluminum oxide ceramics, glasses such as borofloat glasses, silicone, and other synthetic polymers such as polystyrene or polytetrafluorethylene (Teflon™).

In a further embodiment of the invention, at least a part of the surface, board or packaging barriers, in particular of the first surface and/or the second surface may be made of a transparent material, i.e. a light-permeable material. Examples of suitable transparent materials include inter alia glasses or glass-like materials such as window glass, borofloat glasses, quartz glasses, topaz glass, or sapphire glass, as well as synthetic polymers such as polymethylmethacrylate, polycarbonate, polycarbonate, polystyrene, or acryl.

Furthermore, at least a part of the surface, board or packaging barriers, in particular of the first surface and/or the second surface may be elastically deformable. That is, at least a part of the respective surface(s) may be made of an elastically deformable material, for example an elastic membrane. A particularly preferred elastic membrane is made of silicone rubber.

A “reaction chamber” as defined herein above may further comprises a chamber body. The term “chamber body”, as used herein, is understood to denote the solid body surrounding the reaction chamber, which may be formed by the first surface, the second surface, and lateral sidewalls. The first surface, the second surface, and/or one or more of the lateral sidewalls may be integral part(s) of the chamber body. That is, the respective surface(s) being an integral part of the chamber body may be made of the same material as the chamber body. Alternatively, one or more of the first surface, the second surface, and/or one or more lateral sidewalls, respectively, may be made of another material than the chamber body. Within the scope of the present invention, it is thus possible that all four surfaces defining the reaction chamber are made of the same material, that two or three surfaces are made of the same material, whereas the remaining surface(s) is (are) made of different material(s), or that each surface is made of different materials.

The chamber body may preferably be made, at least in part, of an amorphous material, in particular of a transparent material. Suitable materials include inter alia glass, synthetic materials such as polycarbonate (e.g. Macrolon™), nylon, polymethylmethacrylate, and Teflon™, and metals such as high-grade steel, aluminum, and brass. In some embodiments of the invention, the chamber body may be made of electrically conductive material, which is preferably selected from the group consisting of polyamide with 5 to 30% carbon fibers, polycarbonate with 5 to 30% carbon fibers, polyamide with 2 to 20% stainless steel fibers, and polyphenylensulfide with 5 to 40% carbon fibers.

It is also within the scope of the present invention that the reaction chamber of the packaged chip is not designed as a single reaction space but may comprise two or more sub-chambers. This can be achieved by providing the first surface and/or the second surface with one or more additional partitions or cavities, which serve as lateral sidewalls between the two or more sub-chambers. It is preferred that the lateral sidewalls between the two or more sub-chambers are formed by elastic seals. In special embodiments, the partitions on the first surface and/or the second surface may not span the distance between the first surface and the second surface in the non-operated device, that is before the distance between the first surface and the second surface is varied. Accordingly, in the non-operated device the two or more sub-chambers are in fluidic contact with each other. However, if the distance between the first surface and the second surface is reduced, the sub-chambers can be separated. Thus, by varying the distance between said two surfaces the partitions may be operated like valves.

The packaged chip may further comprise one or more means which allow essentially vertical movements of the first surface and/or the second surface relative to each other. The term “vertical movement”, as used herein, denotes a movement of either one or both surfaces of the device perpendicular to their respective surface areas, thus resulting in a variation of the distance between them. A variation of the distance between said two surfaces is understood to include both a reduction and an increase of said distance. A reduction of the distance between the first surface and the second surface of the device can be achieved either by moving the first surface towards the second surface, by moving the second surface towards the first surface or by moving both surfaces towards each other. Vice versa, an increase of the distance between the first surface and the second surface of the device can be achieved either by moving the first surface away from the second surface, by moving the second surface away from the first surface or by moving both surfaces away from each other. The distance between the first surface and the second surface may be varied by applying pressure and/or traction to either one or to both surfaces via said one or more means.

A chip, preferably a packaged chip, manufactured according to the method of the present invention may further comprise one or more means, which, when the distance between the first surface and the second surface is reduced, allow keeping the volume of the reaction chamber essentially constant. That is, compensation zones are provided to which any liquid and/or gaseous material being present in the reaction chamber between the first surface and the second surface can be displaced when the distance between said surfaces is reduced. This may preferably be accomplished by providing a reaction chamber laterally delimited by sidewalls made of an elastic material. According to the present invention, one or more lateral sidewalls can be made of an elastic material. A particularly preferred elastic material is silicone rubber.

An alternative means, which allows keeping the volume of the reaction chamber essentially constant, may comprise a channel that is connected to the reaction chamber of the packaged chip and that is filled with a viscous liquid such as silicon oil. Thus, when the distance between the first surface and the second surface is reduced, the viscous liquid may become displaced in the channel by the excess sample material becoming displaced from the reaction chamber.

In another embodiment, the chip, in particular the packaged chip may further comprise a temperature control unit and/or temperature regulating unit for controlling and/or regulating the temperature within the reaction chamber, for example, in order to achieve optimal reaction conditions, a high sensitivity and/or specificity of reactions or interaction to be carried out. Such a temperature control unit and/or temperature regulating unit may comprise one or more separate heating and/or cooling elements, which may directly contact the first surface and/or the second surface. The one or more heating and/or cooling elements are preferred to be made of a heat conductive material. Examples of such heat conductive materials include inter alia silicon, ceramic materials like aluminum oxide ceramics, and/or metals like high-grade steel, aluminum, copper, or brass. An exemplary detailed description of a temperature control unit and/or temperature regulating unit according to the present invention can also be found in the International Patent Application WO 01/02094.

Controlling/regulating the temperature within the reaction chamber may also be achieved by using a chamber body made of an electrically conductive material. Preferred examples of electrically conductive materials include electrically conductive synthetic materials, such as polyamide with 5 to 30% carbon fibers, polycarbonate with 5 to 30% carbon fibers, polyamide with 2 to 20% stainless steel fibers, and polyphenylene sulfide with 5 to 40% carbon fibers. It is further preferred that the chamber body is designed to comprise swellings and diminutions which allow specific heating of the reaction chamber or the corresponding surfaces. Furthermore, the use of such elements has the advantage that, even when using a material with a comparably low heat conductivity, a homogenous tempering of the reaction chamber is ensured, as heat is released in each such volume element.

Measuring the temperature in the reaction space may be performed by various methods known to the skilled person, for example by using integrated resistance sensors, semi-conductor sensors, light waveguide sensors, polychromatic dyes or liquid crystals. Furthermore, the temperature in the reaction chamber may be determined by using an integrated temperature sensor in the chamber body, a pyrometer or an infrared sensor, or by measuring the temperature-dependent alteration of parameters such as the refraction index at the surface on which detection takes place or the pH value of the sample, for example by measuring the color alteration of a pH-sensitive indicator.

In a further preferred embodiment of the present invention the chip manufactured according to the methods of the present invention is a packaged chip, which may comprise a reaction chamber with inlets for flowing fluid, and an alignment structure for placing the chip at a desired location with respect to a scanner.

The term “inlet”, as used herein, denotes an opening of variable size, preferably of the dimension of the height of the packaged chip, half of the height of packaged chip, 25% of the height of the packaged chip, or most preferably about 10% of the height of the height of the packaged chip. Preferably, the first surface and/or the second surface of the reaction chamber may comprise one or more inlets, e.g. 1, 2, 3, 4 or 5 inlets.

The inlet may allow fluids to enter into and flow through the packaged chip, in particular the reaction chamber of the chip or any further sub-spaces as defined herein above. The term “flowing fluid” means that a fluid, e.g. a reaction medium, buffer etc. may move either driven by capillary forces or by virtue of pressure or driving forces through a packaged chip or reaction chamber. In a specific embodiment, the inlet may be connected to means such as a vacuum pump that allow the application of a vectored vacuum perpendicular or in parallel to the first surface. The application of such vectored vacuum may enable and/or facilitate the vertical diffusion (relative to the first surface) of fluids or substances, e.g. one or more species of capture molecules or the like through the reaction chamber. Typically, the vacuum applied to the reaction chamber is in the range of 1 hPa to 1013 hPa, preferably in the range of 10 hPa to 750 hPa, and particularly preferably in the range of 100 hPa to 500 hPa.

Furthermore each inlet may comprise a seal to retain the fluid within the cavity. Thereby a sealed thermostatically controlled chamber in which fluids can easily be introduced may be provided.

The term “alignment structure for placing the chip at a desired location with respect to a scanner”, as used herein, denotes support structures, e.g. in the form of alignment holes, alignment marks or markings, which may exist at selected locations of the chip, in particular the packaged chip. The alignment structures may be used to mount or position the chip, in particular the packaged chip to an apparatus, e.g., scanner or the like. Preferably, the packaged chip may be asymmetric, e.g. by having asymmetric alignment structures like asymmetric holes, cropped angels, preferably one, two, or three cropped angles. The asymmetry of the packaged chip may be used in order to eliminate malusage or malpositioning of the chip with respect to, e.g. a scanning system. Typically, the device may only be entered into a scanning system if properly placed, i.e. if the asymmetry is detected by the scanning device. The asymmetrical elements of the packaged chip may be adapted to the form and format of scanning devices known to the person skilled in the art.

In a further embodiment of the present invention, the chip, e.g. a packaged chip manufactured according to the present invention, may be used for the detection and measurement of specific parameters. The parameter may mainly depend on the substance deposited and immobilized on the support material and the intended interaction scheme between said substance and, e.g., possible interactors. For instance, such a chip may be used for the performance of assays, e.g. molecular assays. A typical assay, comprised within the scope of the present invention, is a nucleic acid interaction or hybridization assay. In order to carry out such assays, a chip according to the present invention may additionally comprise or be combined or associated with one or more detection systems, e.g. a scanner or scanning device. Furthermore, an assay may be carried out based on such detection systems. The term “associated” means that the chip or packaged chip may be transfer from one place, e.g. a place where an assay is carried out or where reaction medium is filled in, to a different place where a detection or scanning process is carried out.

Typically, a corresponding detection system or scanner is connected or associated to the reaction chamber. Preferably, the detection system may be positioned opposite to the first surface and/or the second surface, on which detection take(s) place. Various optical and non-optical detection systems or scanners are well established in the art and may appropriately be used. A general description of detection methods that can be used with the invention may be derived, for example, from Lottspeich, F., and Zorbas H. (1998) Bioanalytik, Spektrum Akademischer Verlag, Heidelberg/Berlin, Germany, in particular from chapters 23.3 and 23.4.

A detection system according to the present invention may preferably be an optical detection system or scanner, in particular a fluorescence-optical detection system. In general, the use of a packaged chip of the present invention in an assay may be based on the measurement of parameters such as fluorescence, optical absorption, resonance transfer, and the like. Preferred systems for the detection of molecular interactions are based on the comparison of the fluorescence intensities of spectrally excited analytes labeled with fluorophores. Fluorescence is the capacity of particular molecules to emit their own light when excited by light of a particular wavelength resulting in a characteristic absorption and emission behavior. In particular, quantitative detection of fluorescence signals is performed by means of modified methods of classical fluorescence microscopy (for review see, e.g., Lichtman, J. W., and Conchello, J. A. (2005) Nature Methods 2, 910-919; Zimmermann, T. (2005) Adv. Biochem. Eng. Biotechnol. 95, 245-265). Thereby, the signals resulting from light absorption and light emission, respectively, are separated by one or more filters and/or dichroites and imaged on suitable detectors such as two-dimensional CCD arrays. Data analysis may be performed by means of digital image processing.

Another optical detection system that may also be used when performing the present invention is confocal fluorescence microscopy, wherein the object is illuminated in the focal plane of the lens via a point light source. Importantly, the point light source, object and point light detector are located on optically conjugated planes. Examples of such confocal systems are described, e.g., in Diaspro, A. (2002) Confocal and 2-photon-microscopy: Foundations, Applications and Advances, Wiley-Liss, Hobroken, N.J. The fluorescence-optical system of the present invention is particularly preferred to represent a fluorescence microscope without an autofocus, for example a fluorescence microscope having a fixed focus.

In alternative chips, in particular packaged chips, according to the present invention means for performing an electrochemical detection of the analytes are provided, for example by measuring the alteration of redox potentials via electrodes connected to the first surface and/or the second surface (see, e.g., Zhu, X. et al. (2004) Lab Chip. 4, 581-587) or by cyclic voltometry (see, e.g., Liu, J. et al. (2005) Anal. Chem. 77, 2756-2761; and Wang, J. (2003) Anal. Chem. 75, 3941-3945). Furthermore, it is also possible to provide means for performing an electric detection, for example by impedance measurement (see, e.g., Radke, S. M. et al. (2005) Biosens. Bioelectron. 20, 1662-1667).

In another embodiment, a chip or packaged chip manufactured according to the present invention may be used for the analysis of biological fluids or liquids like blood, urine, a cell extract, a tissue extract, a tissue exudate, lymph fluid, sputum, saliva or cerebrospinal fluids in order to detect the presence of pathogens etc. or for the detection of the presence of disease states. The term “detection” relates to the employment of a chip or packaged chip manufactured according to the present invention for interaction reactions with substances, e.g. nucleic acids or oligonucleotides, proteins etc. derived from different sources, tissues, samples, organs etc. linked to medical or biological identification purposes described herein below. Preferably, such substances derived from different sources may be labeled, e.g. with labels as defined herein above, before they are brought into contact with, or the vicinity of a chip or packaged chip as defined herein above in order to allow a recognition of a specific interaction or hybridization between a nucleic acid immobilized in the array and a target nucleic acid derived from any of the above mentioned sources. The preparation and/or processing of such target substances is known to the person skilled in the art and may be derived, for example, from a textbook like Sambrook et al., Molecular Cloning: A Laboratory Manual, 2001, Cold Spring Harbor Laboratory Press.

Preferably, the chip or packaged chip or any assay based on the chip or packaged chip of the present invention may be used for the detection and/or diagnosis of deficiencies or disorders of the immune system, e.g. the proliferation, differentiation, or mobilization (chemotaxis) of immune cells. Immune cells develop through a process called hematopoiesis, producing myeloid (platelets, red blood cells, neutrophils, and macrophages) and lymphoid (B and T lymphocytes) cells from pluripotent stem cells. The etiology of these immune deficiencies or disorders may be genetic, somatic, such as cancer or some autoimmune disorders, acquired (e.g., by chemotherapy or toxins), or infectious. In another preferred embodiment a chip or packaged chip as defined herein above may be useful in detecting deficiencies or disorders of hematopoictic cells. Examples of immunologic deficiency syndromes include, but are not limited to: blood protein disorders (e.g. agammaglobulinemia, dysgammaglobulinemia), ataxia telangiectasia, common variable immunodeficiency, Digeorge Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

Moreover, a chip or packaged chip or any assay based on the chip or packaged chip of the present invention could also be used to monitor hemostatic or thrombolytic activity. For example, the chip or packaged chip may be used to detect blood coagulation disorders (e.g. afibrinogenemia, factor deficiencies) or blood platelet disorders (e.g. thrombocytopenia). Furthermore, the chip or packaged chip may be used to determine parameters indicative for a high risk of heart attacks (infarction) or strokes or detect pre-infarction parameters; such parameter are known to the person skilled in the art.

A chip or packaged chip of the present invention could also be used for the detection and/or diagnosis of autoimmune disorders. Examples of autoimmune disorders that can be detected and/or diagnosed include, but are not limited to: Addison's Disease, hemolytic anemia, antiphospholipid syndrome, rheumatoid arthritis, dermatitis, allergic encephalomyelitis, glomerulonephritis, Goodpasture's-Syndrome, Graves Disease, Multiple Sclerosis, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin dependent diabetes mellitis, and autoimmune inflammatory eye disease.

Similarly, a predisposition for allergic reactions and conditions, such as asthma (particularly allergic asthma) or other respiratory problems, may also be detected and/or diagnosed with a chip or packaged chip as defined herein above.

Moreover, the chip or packaged chip of the present invention may be used for the detection and/or diagnosis of hyperproliferative disorders, including neoplasms. Examples of hyperproliferative disorders that can be detected include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital. Further examples of hyperproliferative disorders that may be detected by using a chip or packaged chip of the present invention include hypergammaglobulinemia, lymphoproliferative disorders, paraproteinemi as, purpura, sarcoidosis, Sezary Syndrome, Waldenstron's Macroglobulinermia, Gaucher's Disease, histiocytosis, and any other hyperproliferative disease, besides neoplasia, located in an organ system listed above.

The chip or packaged chip of the present invention may also be used to detect infectious agents or to detect and/or diagnose infections. Viruses are one example of an infectious agent that can cause diseases or symptoms that can be detected by the chip or packaged chip of the present invention. Examples of viruses, include, but are not limited to the following DNA and RNA viral families: Arbovirus, Adenoviridae, Arenaviridae, Arterivirus, Bimaviridae, Bunyaviridae, Caliciviridae, Circoviridae, Coronaviridae, Flaviviridae, Hepadnaviridae (Hepatitis), Herpesviridae (such as Cytomegalovirus, Herpes Simplex, Herpes Zoster), Mononegavirus (e.g. Paramyxoviridae, Morbillivirus, Rhabdoviridae), Orthomyxoviridae (e.g. Influenza), Papovaviridae, Parvoviridae, Picornaviridae, Poxyiridae (such as Smallpox or Vaccinia), Reoviridae (e.g. Rotavirus), Retroviridae (HTLV-I, HTLV-II, Lentivirus), and Togaviridae (e.g. Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g., Kaposi's, warts), and viremia.

Similarly, the chip or packaged chip of the present invention may be used to detect bacterial or fungal agents that can cause disease or symptoms including, but not limited to the following Gram-Negative and Gram-positive bacterial families and fungi: Actinomycetales (e.g. Corynebacterium, Mycobacterium, Norcardia), Aspergillosis, Bacillaceae (e.g. Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeria, Mycoplasmatales, Neisseriaceae (e.g. Acinetobacter, Gonorrhea, Menigococcal), Pasteurellacea Infections (e.g. Actinobacillus, Heamophilus, Pasteureila), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic-infections (e.g. AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g. cellulitis, dermatocycoses), toxemia, urinary tract infections, wound infections.

In a particularly preferred embodiment the chip or packaged chip of the present invention may be used to detect the following pathogens or their presence in samples of the human or animal body or samples of human or animal excrementa: Escherichia coli, Staphylococcus epidermidis, Staphylococcus aureus, Enterococcus faecalis, Klebsiella pneumoniae, Pseudomonas aeruginosa, Enterococcus faecium, Streptococcus pneumoniae, Staphylococcus capitis, Klebsiella oxytoca, Streptococcus agalactiae, Proteus mirabilis, Staphylococcus cohnii, Staphylococcus haemolyticus, Acinetobacter baumannii, Enterococcus sp., Proteus vulgaris, Serratia marcescens, Staphylococcus warneri, Staphylococcus hominis, Streptococcus anginosus, Streptococcus mitis, Staphylococcus auricularis, Staphylococcus lentus, Streptococcus beta haem Group G, Streptococcus beta haem Group F, Streptococcus gordonii, Streptococcus Group D, Streptococcus oxalis, Streptococcus parasanguis, Streptococcus salivarius, Citrobacter freudii, Listeria monocytogenes, Micrococcus luteus, Acinetobacter junii, Bacillus cereus, Bacteroides caccae, Bacteroides uniformis, Bacteroides vulgatus, Clostridium perfringens, Corynebacterium pseudodiphtheriticum, Corynebacterium sp., Corynebacterium urealyticum, Fusiobacterium nucleatum, Micrococcus sp., Pasteurella multocida, Propionibacterium acnes, Ralstonia pickettii, Salmonella ser. Paratyphi B and Yersinia enterocditi.

Moreover, the chip or packaged chip of the present invention may be used to detect parasitic agents causing disease or symptoms including, but not limited to, the following families: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, and Trichomonas. These parasites can cause a variety of diseases or symptoms, including, but not limited to: Scabies, Trombiculiasis, eye infections, intestinal disease (e.g. dysentery, giardiasis), liver disease, lung disease, opportunistic infections (e.g. AIDS related), Malaria, pregnancy complications, and toxoplasmosis, which may also be detected by use of the chip or packaged chip of the present invention.

The following examples and figures are provided for illustrative purposes. It is thus understood that the example and figures are not to be construed as limiting. The skilled person in the art will clearly be able to envisage further modifications of the principles laid out herein.

### EXAMPLES

### Example 1

### Deposition Assay

The experiment was performed with a Biodyne C membrane. First the membrane was locally activated by printing on a selective number of spots a 16% EDC solution (transfer solution). The pattern is depicted in FIG. 5. For every spot, 8 droplets of 120 pl each were printed.

Subsequently, on the same substrate, a substance solution comprising a fluorescently labeled oligonucletide in a concentration of 10 μM was printed on the membrane. For the substance solution the same volume was used as for the transfer solution. As can be derived from FIG. 6, spots were placed not only on the activated spots, but also on the columns in between.

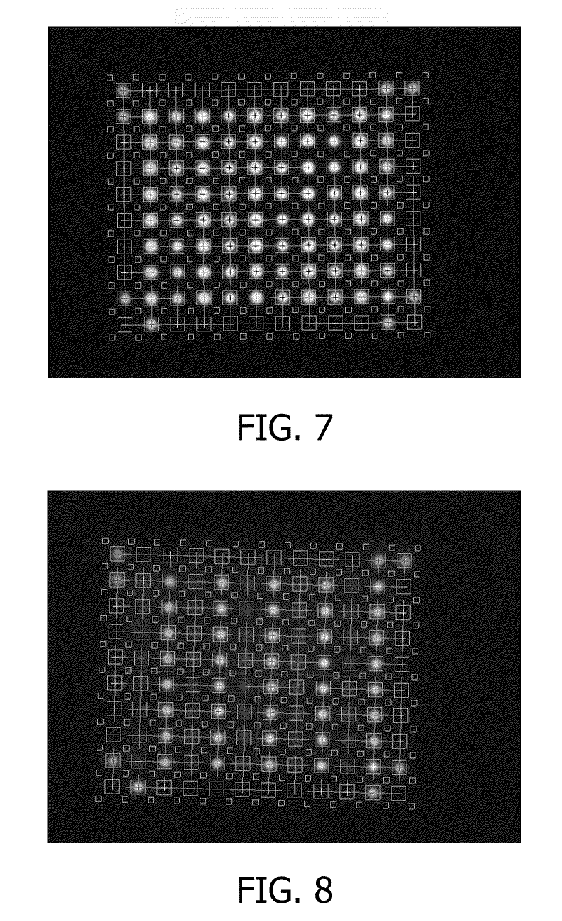

Subsequently, a picture was made by imaging the fluorescently labeled spots directly after printing. As can be derived from FIG. 7 the intensity of the spots is the same, which is expected as the same number of fluorophores has been deposited on each spot. However, the spots where the transfer fluid has been printed are smaller.

### Example 2

### Blocking and Washing

In order to prove the immobilization is stable and that non-immobilized residual material may be removed the substrate was blocked and washed after printing. In particular, all material that was not immobilized on the surface was removed. This was done by incubation with 0.1M NaOH. Afterwards the membranes were shortly rinsed with milliQ water. Then a 6 minute wash was performed in 2×SSPE and 0.1% SDS. Subsequently the membranes were washed in 20 mM EDTA, pH 8.0, and dried with dry nitrogen.

As can be derived from FIG. 8, which shows the image of the same membrane measured after washing, the spots where the transfer fluid has been printed are clearly visible whereas the spots where no transfer fluid has been printed are hardly visible. This means that only on the spots where the transfer fluid was deposited, the substance has bound.

The result is, thus, a proof of principle that the method for depositing a substance as nucleic acids is workable and efficient.

1. A method for depositing a substance on a support, comprising the steps of:
(a) providing a substance solution;
(b) providing a transfer solution capable of activating the support;
(c) depositing said transfer solution on a predefined position of the support; and
(d) depositing said substance solution on the same predefined position where the transfer solution was placed, whereby an immobilization of the deposited substance at the location of overlap between the deposited transfer solution and the deposited substance solution on said support is achieved,
with the proviso that said transfer solution and said substance solution are not placed together or as a mixed solution on the support.

2. The method of claim 1, wherein the interim between deposition step (c) and (d) is a predefined, fixed period of time.

3. The method of claim 1, wherein the deposition of the substance solution of step (d) is carried out before the deposition of the transfer solution of step (c).

4. The method of claim 1, wherein said support comprises amine-reactive groups.

5. The method of claim 4, wherein said support comprises carboxylic groups.

6. The method of claim 1, wherein said support is a porous substrate like nylon or a non-porous substrate like glass, poly-L-lysine coated material, nitrocellulose, polystyrene, cyclic olefin copolymer (COC), cyclic olefin polymer (COP), polypropylene, polyethylene or polycarbonate.

7. The method of claim 1, wherein said activation of the support is a chemical activation.

8. The method of claim 7, wherein said transfer solution comprises chemical moieties capable of reacting with amine groups or carboxylic groups.

9. The method of claim 8, wherein said transfer solution comprises EDC (1-ethyl-3-(3-dimethylaminopropyl) carbodiimide hydrochloride) or NHS (N-hydrosuccinimide) or a mixture of EDC and NHS.

10. The method of claim 1, wherein said substance solution comprises a nucleic acid, a protein or a sugar, or a modified derivative thereof, or any combination thereof, preferably a nucleic acid, protein or sugar, or modified derivative thereof comprising an amine group.

11. The method of claim 1, wherein in a further step (e) the support is washed, whereby substance solution, which is not fixated according to step (d), is removed.

12. Use of a method as defined in claim 1 for the manufacturing of a chip.

13. A method for manufacturing a chip, wherein a substance is deposited on a chip substrate according to the method of claim 1.

14. A chip manufactured according to the method of claim 13.

15. The chip of claim 14, which is a packaged chip comprising a reaction chamber with inlets for flowing fluid, and alignment structures for placing the chip at a desired location with respect to a scanner.
